(19) United States
(12) Patent Application Publication
(10) Pub. No.: US 2026/0146082 A1
(43) Pub. Date: May 28, 2026

(54) BISPECIFIC ANTI-MYOSTATIN AND ACTIVIN A ANTIBODY OR ANTIGEN BINDING FRAGMENTS THEREOF

(71) Applicants: iBio, Inc., San Diego, CA (US); AstralBio, Inc., Boston, MA (US)

(72) Inventors: Cory Schwartz, San Diego, CA (US); Matthew William Dent, San Diego, CA (US); Tam Thi Thanh Phuong, San Diego, CA (US); Tom Sih-Yuan Hsu, San Diego, CA (US); Cody Allen Moore, San Diego, CA (US); Alexander Tomoaki Taguchi, San Diego, CA (US); Martin Brenner, San Diego, CA (US)

(21) Appl. No.: 19/399,960

(22) Filed: Nov. 25, 2025

Related U.S. Application Data
(60) Provisional application No. 63/725,707, filed on Nov. 27, 2024.

(51) Int. Cl. C07K 16/22

(52) U.S. Cl. CPC C07K 16/22; C07K 2317/31; C07K 2317/76

(57) ABSTRACT

Provided herein are novel bispecific anti-myostatin and anti-Activin A antibody or antigen binding fragments thereof comprising: a first antibody or antigen binding fragments thereof that binds to myostatin; and a second antibody or antigen binding fragments thereof that binds to Activin A.

### CROSS-REFERENCE TO RELATED APPLICATIONS

This application claims priority to U.S. Provisional Application Ser. No. 63/725,707, filed Nov. 27, 2024, the entire contents of which are incorporated herein by reference.

### TECHNICAL FIELD

This document relates to materials and methods for triggering an immune response, and particularly to the use of novel bispecific, anti-Myostatin and Activin A antibody or antigen binding fragments thereof to trigger immune activation or tolerance, and to treat autoimmune diseases, cancer, antimicrobial, antiviral, and other immune responses.

### REFERENCE TO A SEQUENCE LISTING

The application contains a Sequence Listing which has been submitted electronically in .XML format and is hereby incorporated by reference in its entirety. Said .XML copy, created on Nov. 24, 2025, is named “IBIO1045.xml” and is 39,371 bytes in size. The sequence listing contained in this .XML file is part of the specification and is hereby incorporated by reference herein in its entirety.

### BACKGROUND

Without limiting the scope of the invention, its background is described in connection with bispecific antibodies.

Antibodies against Activin A are taught by Han, et al., in U.S. Pat. No. 11,542,325, entitled, “Anti-Activin A Antibodies and Uses Thereof”. These inventors are said to teach anti-activin A binding proteins, including antibodies. In particular embodiments, the disclosure provides fully human, humanized, and chimeric anti-Activin A antibodies that bind human activin A, Activin A-binding fragments and derivatives of such antibodies, and Activin A-binding polypeptides comprising such fragments. Other embodiments are said to include nucleic acids encoding such antibodies, antibody fragments and derivatives and polypeptides, cells comprising such polynucleotides, methods of making such antibodies, antibody fragments and derivatives and polypeptides, and methods of using such antibodies, antibody fragments and derivatives and polypeptides, including methods of treating or diagnosing subjects having activin A-related disorders or conditions including cachexia related to gonadal cancer, other cancers, rheumatoid arthritis, and other diseases.

One such patent is U.S. Pat. No. 11,155,611, issued to Scholar Rock and U.S. Patent Publication Nos. 20180344844, 20170333558, and 20170198032, which are directed to compositions and methods for making and using anti-myostatin antibodies. These inventors are said to teach antibodies, or antigen-binding fragments thereof, that specifically bind proMyostatin and/or latent Myostatin, and methods and uses thereof for treating metabolic diseases.

Another such patent application is U.S. Patent Publication No. 20190002548, filed by Ruike and Kuramochi, entitled “Anti-Myostatin Antibodies and Methods of Use”. These applicants are said to teach an anti-myostatin antibody that binds to mature myostatin, and uptake of the antibody into cells is enhanced when complexed with the antigen. The invention also provides isolated nucleic acids encoding an anti-myostatin antibody, host cells comprising a nucleic acid encoding the antibody, and a method of producing the antibody. The anti-myostatin are said to be used as a medicament for treating a muscle wasting disease, increasing mass of muscle tissue, and increasing strength of muscle tissue.

Despite these advances, novel bispecific antibodies are needed that have unique binding characteristics, target specific epitopes, and can be used for treating disorders related to cachexia, cancers, arthritis and other diseases or conditions.

### SUMMARY

As embodied and broadly described herein, an aspect of the present disclosure relates to a bispecific anti-myostatin and anti-Activin A antibody or antigen binding fragments thereof, comprising: a first antibody or antigen binding fragments thereof that binds to myostatin; a second antibody or antigen binding fragments thereof that binds to Activin A; wherein the first and second antibody or antigen binding fragments are selected to modulate or bind to both myostatin and anti-Activin A. In one aspect, the anti-myostatin antibody or binding fragment thereof comprising: a heavy chain variable domain (VH) complementarity determining region 1 (CDR1) comprising the amino acid sequence of any one of the following SEQ ID NO: 3; a VH CDR2 of SEQ ID NO: 4; and a VH CDR3 of SEQ ID NO: 5; and a light chain variable domain (VL) CDR1 of SEQ ID NO: 6; a VL CDR2 of SEQ ID NO: 7; and a VL CDR3 of SEQ ID NO: 8. In another aspect, the anti-Activin A antibody or binding fragment thereof comprising: a heavy chain variable domain (VH) complementarity determining region 1 (HCDR1) comprising the amino acid sequence of SEQ ID NO: 13; a HCDR2 of SEQ ID NO: 14; and a HCDR3 of SEQ ID NO: 15; and a light chain variable domain (VL) complementarity determining region 1 (LCDR1) comprising the amino acid sequence of SEQ ID NO: 16; LCDR2 of SEQ ID NO: 17; and LCDR3 of SEQ ID NO: 18. In another aspect, the anti-myostatin antibody or binding fragment thereof comprises: a heavy chain having at least 80%, or at least 85%, or at least 90%, 91%, 92%, 93%, 94%, 95%, 96%, 97%, 98%, 99%, or 100% sequence identity to SEQ ID NO: 1, and a light chain having at least 80%, or at least 85%, or at least 90%, 91%, 92%, 93%, 94%, 95%, 96%, 97%, 98%, 99%, or 100% sequence identity to SEQ ID NO: 2. In another aspect, the anti-Activin A antibody or binding fragment thereof comprises: a heavy chain having at least 80%, or at least 85%, or at least 90%, 91%, 92%, 93%, 94%, 95%, 96%, 97%, 98%, 99%, or 100% sequence identity to SEQ ID NO: 19, and a light chain having at least 80%, or at least 85%, or at least 90%, 91%, 92%, 93%, 94%, 95%, 96%, 97%, 98%, 99%, or 100% sequence identity to SEQ ID NO: 20. In another aspect, the bispecific antibody or binding fragment thereof is encoded by a nucleic acid that has at least 80%, or at least 85%, or at least 90%, 91%, 92%, 93%, 94%, 95%, 96%, 97%, 98%, 99%, or 100% sequence identity to SEQ ID NOS: 21, 22, 23, and 24. In another aspect, the bispecific antibody or binding fragment thereof is encoded by a nucleic acid that has at least 80%, or at least 85%, or at least 90%, 91%, 92%, 93%, 94%, 95%, 96%, 97%, 98%, 99% or 100% to SEQ ID NOS: 29 and 30. In another aspect, the bispecific antibody or binding fragment thereof is encoded by a nucleic acid that has at least 80%, or at least 85%, or at least 90%, 91%, 92%, 93%, 94%, 95%, 96%, 97%, 98%, 99% or 100% to SEQ ID NOS: 9 or 19, and a VL nucleic acid that has at least 80%, or at least 85%, or at least 90%, 91%, 92%, 93%, 94%, 95%, 96%, 97%, 98%, 99% or 100% to SEQ ID NOS: 10 or 20. In another aspect, the bispecific antibody or binding fragment thereof is encoded by a nucleic acid that has at least 80%, or at least 85%, or at least 90%, 91%, 92%, 93%, 94%, 95%, 96%, 97%, 98%, 99% or 100% to SEQ ID NOS: 26 and 26; 27 and 28; or 31 and 32. In another aspect, the Fc domain of any one of the following: human IgG1, human IgG2, human IgG3, human IgG4, or mutants thereof. In another aspect, the bispecific antibody comprises a bispecific knob-in-hole, IgG-ScFv, ScFv-IgG, scFV-Fc-ScFv, or scFv-scFv-Fc. In another aspect, the bispecific antibody or antigen binding fragments further comprises a linker between the first and second antibody or antigen binding fragments thereof. In another aspect, the bispecific antibody or antigen binding fragments further comprises a payload. In another aspect, the bispecific antibody or antigen binding fragments further comprises an anti-cancer drug.

As embodied and broadly described herein, an aspect of the present disclosure relates to a polynucleotide encoding the anti-myostatin and anti-Activin A antibody or antigen binding fragments described hereinabove.

As embodied and broadly described herein, an aspect of the present disclosure relates to a n expression vector comprising a nucleic acid encoding bispecific anti-myostatin and anti-Activin A antibody or antigen binding fragments described hereinabove.

As embodied and broadly described herein, an aspect of the present disclosure relates to a host cell comprising the vector described hereinabove.

As embodied and broadly described herein, an aspect of the present disclosure relates to a method of making a bispecific anti-myostatin and anti-Activin A antibody or antigen binding fragments comprising: culturing a cell comprising the polynucleotide described hereinabove, in a culture medium, expressing the polynucleotide, and collecting the bispecific anti-myostatin and anti-Activin A antibody or antigen binding fragments thereof from the cells or culture medium.

As embodied and broadly described herein, an aspect of the present disclosure relates to a method of treating a disease or condition of obesity, muscle-related, bone growth, cachexia, or cancer in a subject, comprising: administering a bispecific anti-myostatin and anti-Activin A antibody or antigen binding fragments described hereinabove, or the polynucleotide described hereinabove, or the expression vector described hereinabove to the patient. In one aspect, the disease or condition is at least one of: obesity, adipose tissue disorder, metabolic syndrome, hyperphagia-associated obesity, dyslipidemia, diabetes, visceral adiposity, sarcopenic obesity, or cachexia in obese patients. In another aspect, the disease is selected from loss of body weight, loss of muscle mass, or loss of fat mass. In another aspect, the cancer is selected from: gonadal tumor, ovarian cancer, benign prostatic hyperplasia, prostate intraepithelial neoplasia, or prostate cancer, or wherein the tumor is bladder cancer, Wilm's tumor, pancreatic cancer, breast cancer, bone cancer, lung cancer, colorectal cancer, cervical cancer, synovial sarcoma, vasoactive intestinal peptide secreting tumors, glioblastoma, medulloblastoma, head and neck squamous cell cancer, oral cancer, oral leukoplakia, anal cancer, esophageal cancer, gastric cancer, bone cancer, or metastatic cancer. In another aspect, the subject is human. In another aspect, the bispecific antibody or antigen binding fragment having a predetermined activity is used during a first phase of treatment, and a second bispecific antibody or antigen binding fragment having a predetermined activity is used during a second phase of treatment, and optionally using one or more different bispecific antibody or antigen binding fragment having a predetermined activity is used during a first phase of treatment for any additional phases of treatment. In another aspect, the bispecific anti-myostatin and anti-Activin A antibody or antigen binding fragments described hereinabove further comprises a payload. In another aspect, the bispecific anti-myostatin and anti-Activin A antibody or antigen binding fragments described hereinabove further comprise an anti-cancer drug. In another aspect, the disease or condition of bone growth is selected from at least one of: metabolic disorders selected from type 2 diabetes, impaired glucose tolerance, syndrome X, insulin resistance induced by trauma, burns or nitrogen imbalance; adipose tissue disorders (obesity); muscle and neuromuscular disorders such as muscular dystrophy (Duchenne muscular dystrophy); amyotrophic lateral sclerosis (ALS); muscle atrophy; organ atrophy; frailty; carpal tunnel syndrome; congestive obstructive pulmonary disease; and sarcopenia, cachexia and other muscle wasting syndromes; osteoporosis, osteoporosis in elderly and/or postmenopausal women; glucocorticoid-induced osteoporosis; osteopenia;

osteoarthritis; and osteoporosis-related fractures; low bone mass due to chronic glucocorticoid therapy, premature gonadal failure, androgen suppression, vitamin D deficiency, secondary hyperparathyroidism, nutritional deficiencies, and anorexia nervosa. In another aspect, the muscle-related diseases or conditions are selected from at least one of: neuromuscular disorders, muscular dystrophy and muscle atrophy, congestive obstructive pulmonary disease, muscle wasting associated with COPD, muscle wasting syndrome, sarcopenia, cachexia, adipose tissue disorders, type 2 diabetes, bone degenerative disease, osteoporosis; musculodegenerative and neuromuscular disorders, tissue repair, wound healing, neurodegenerative diseases, amyotrophic lateral sclerosis, immunologic disorders, disorders related to abnormal proliferation or function of lymphocytes, and obesity or disorders related to abnormal proliferation of adipocytes.

As embodied and broadly described herein, an aspect of the present disclosure relates to a method of selecting a bispecific anti-myostatin and anti-Activin A antibody or antigen binding fragments comprising: selecting an antibody against the antigen; generating the bispecific anti-myostatin and anti-Activin A antibody or antigen binding fragments; contacting the bispecific anti-myostatin and anti-Activin A antibody or antigen binding fragments under conditions in which a muscle cell expresses myostatin and Activin A; measuring the effect of the bispecific anti-myostatin and anti-Activin A antibody or antigen binding fragments on the muscle cell; and selecting the combination of bispecific anti-myostatin and anti-Activin A antibody or antigen binding fragments having a predetermined activity level. In one aspect, the bispecific antibody comprises bispecific knob-in-hole, IgG-ScFv, ScFv-IgG, scFV-Fc-ScFv, or scFv-scFv-Fc. In another aspect, the activity is selected from at least one of: cachexia, loss of body weight, loss of muscle mass, or loss of fat mass; gonadal tumor, ovarian cancer, benign prostatic hyperplasia, prostate intraepithelial neoplasia, or prostate cancer, or wherein the tumor is bladder cancer, Wilm's tumor, pancreatic cancer, breast cancer, bone cancer, lung cancer, colorectal cancer, cervical cancer, synovial sarcoma, vasoactive intestinal peptide secreting tumors, glioblastoma, medulloblastoma, head and neck squamous cell cancer, oral cancer, oral leukoplakia, anal cancer, esophageal cancer, gastric cancer, bone cancer, or metastatic cancer; wherein the disease or condition of bone growth is selected from at least one of: metabolic disorders selected from type 2 diabetes, impaired glucose tolerance, syndrome X, insulin resistance induced by trauma, burns or nitrogen imbalance; adipose tissue disorders (obesity); muscle and neuromuscular disorders such as muscular dystrophy (Duchenne muscular dystrophy); amyotrophic lateral sclerosis (ALS); muscle atrophy; organ atrophy; frailty; carpal tunnel syndrome; congestive obstructive pulmonary disease; and sarcopenia, cachexia and other muscle wasting syndromes; osteoporosis, osteoporosis in elderly and/or postmenopausal women; glucocorticoid-induced osteoporosis; osteopenia; osteoarthritis; and osteoporosis-related fractures; low bone mass due to chronic glucocorticoid therapy, premature gonadal failure, androgen suppression, vitamin D deficiency, secondary hyperparathyroidism, nutritional deficiencies, and anorexia nervosa; or wherein the muscle-related diseases or conditions are selected from at least one of: neuromuscular disorders, muscular dystrophy and muscle atrophy, congestive obstructive pulmonary disease, muscle wasting associated with diabetes, bone degenerative disease, osteoporosis; musculodegenerative and neuromuscular disorders, tissue repair, wound healing, neurodegenerative diseases, amyotrophic lateral sclerosis, immunologic disorders, disorders related to abnormal proliferation or function of lymphocytes, and obesity or disorders related to abnormal proliferation of adipocytes. In another aspect, the bispecific anti-myostatin and anti-Activin A antibody or antigen binding fragments thereof are selected from: a heavy chain variable domain (VH) complementarity determining region 1 (CDR1), CDR2, and CDR3 comprising the amino acid sequence of any one of the following SEQ ID NOs: 3, 4, 5; and 13, 14, 15; and a light chain variable domain (VL) CDR1, CDR2, and CDR3 comprising the amino acid sequence of any one of the following SEQ ID NOs: 6, 7 and 8; and 16, 17, and 18. In another aspect, the bispecific anti-myostatin and anti-Activin A antibody or antigen binding fragments thereof comprises: a heavy chain comprises at least 80%, or at least 85%, or at least 90%, 91%, 92%, 93%, 94%, 95%, 96%, 97%, 98%, 99% or 100% to SEQ ID NOs: 1 and 2, and a light chain comprises at least 80%, or at least 85%, or at least 90%, 91%, 92%, 93%, 94%, 95%, 96%, 97%, 98%, 99% or 100% to SEQ ID NOs: 11 and 12. In another aspect, the bispecific anti-myostatin and anti-Activin A antibody or antigen binding fragments antibody or binding fragment thereof is encoded by a nucleic acid that comprises: a VH comprising a nucleic acid sequence that comprises at least 80%, or at least 85%, or at least 90%, 91%, 92%, 93%, 94%, 95%, 96%, 97%, 98%, 99% or 100% to SEQ ID NOS: 9 and 19, and a VL comprising a nucleic acid sequence that comprises at least 80%, or at least 85%, or at least 90%, 91%, 92%, 93%, 94%, 95%, 96%, 97%, 98%, 99% or 100% to SEQ ID NOs: 10 and 20. In another aspect, the bispecific anti-myostatin and anti-Activin A antibody or antigen binding fragments comprises: a heavy chain variable domain (VH) comprising SEQ ID NOS: 21, 23, or 29; and a light chain variable domain (VL) comprising SEQ ID NO: 22, 24, and 30.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The present application can be understood by reference to the following description taken in conjunction with the accompanying figures.

FIGS. 1A and 1B are representations of two examples of the bispecific antibody constructs of the present disclosure using that anti-activin A antibody A9 and the anti-myostatin antibody L1C9 binding domains. FIG. 1A shows a bifunctional A9xL1C9kih that includes Knob in Hole Fc domains. FIG. 1B shows a bifunctional A9xL1C9FiS that is an IgG-ScFv.

FIGS. 2A to 2C are graphs comparing the activity of the anti-activin A antibody A9 and the anti-myostatin antibody L1C9 binding domains and the bifunctional constructs. FIG. 2A: Reporter cell line to measure GDF8 antagonism. FIG. 2B: Reporter cell line to measure Activin A antagonism. FIG. 2C: Reporter cell line to measure combined GDF8 and Activin A antagonism.

FIGS. 3A to 3D are graphs comparing the activity of the anti-activin A antibody A9 and the anti-myostatin antibody L1C9 binding domains and the bifunctional constructs. FIG. 3A: Myotube coverage as measure of myoblast differentiation. GDF8 antagonism. FIG. 3B: Myotube coverage as measure of myoblast differentiation. GDF11 antagonism. FIG. 3C: Myotube coverage as measure of myoblast differentiation. Activin A antagonism. FIG. 3D: Myotube coverage as measure of myoblast differentiation. Combined GDF8, GDF11, and Activin A antagonism.

FIGS. 4A to 4D are graphs comparing the activity of the anti-activin A antibody A9 and the anti-myostatin antibody L1C9 binding domains and the bifunctional constructs. FIG. 4A: Fusion index as measure of myoblast differentiation. GDF8 antagonism. FIG. 4B: Fusion index as measure of myoblast differentiation. GDF11 antagonism. FIG. 4C: Fusion index as measure of myoblast differentiation. Activin A antagonism. FIG. 4D: Fusion index as measure of myoblast differentiation. Combined GDF8, GDF11, and Activin A antagonism.

### DETAILED DESCRIPTION

While the making and using of various aspects of the present disclosure are discussed in detail below, it should be appreciated that the present disclosure provides many applicable inventive concepts that can be embodied in a wide variety of specific contexts. The specific aspects discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific aspects of the disclosure, but their usage does not delimit the disclosure, except as outlined in the claims.

It should be understood that, unless clearly indicated, in any method described or disclosed herein that includes more than one act, the order of the acts is not necessarily limited to the order in which the acts of the method are recited, but the disclosure encompasses exemplary embodiments in which the order of the acts is so limited.

### Muscle Conditions and Disorders.

In some aspects, the methods of the present invention are suitable for treating or preventing muscle conditions and disorders. As used herein, the term “muscle condition” or “muscle disorder” refers to a disease, condition, or disorder, where the muscle does not function normally, or a disease, condition, or disorder, where the function of muscle is normal, but there is less force generated by the muscle due to a reduced amount of muscle available. A muscle condition or disorder may include, without limitation, a myopathy, muscular atrophy, a muscular dystrophy, etc. Such conditions may be caused by a defect or defects in a motor neuron, a genetic mutation, or an injury, such as a nerve injury.

In another aspect, the muscle condition is a myopathy. As used herein, the term “myopathy” refers to a muscular condition characterized by impaired muscle structure or function, typically resulting in muscular weakness; a muscular condition characterized by normal muscle structure but impaired or abnormal neuronal input, which in turn affects muscle function; or inflammatory myopathies and/or autoimmune myopathies, e.g., myasthenia gravis.

Myopathies for treatment with the antibodies or antigen binding fragments can also include muscular conditions that are neuromuscular or musculoskeletal in nature. The myopathy can be an inherited myopathy such as, e.g., dystrophies, myotonias, congenital myopathies (nemaline myopathy, multi/minicore myopathy, or centronuclear myopathy), mitochondrial myopathies, familial periodic myopathies, inflammatory myopathies and metabolic myopathies (glycogen storage diseases and lipid storage disorder). In some aspects, the myopathy is an acquired myopathy, such as, e.g., external substance induced myopathy (drug-induced myopathy and glucocorticoid myopathy, alcoholic myopathy, and myopathy due to other toxic agents), myositis (dermatomyositis, polymositis and inclusion body myositis), myositis ossificans, rhabdomyolysis, and myoglobinurias, and disuse atrophy. In some aspects, the myopathy is disuse atrophy, which may be caused by prolonged disuse of muscles, leading to deterioration of normal muscle function, such as e.g., atrophy resulting from hospitalization, bone fracture(s), or by nerve injury. In some aspect the myopathy is related to a disease or disorder such as amyotrophic lateral sclerosis (ALS), spinal muscular atrophy (SMA), cachexia syndromes due to renal failure, cardiac conditions, long-COVID, and/or cancer. In some aspect, the myopathy is related to ageing, sarcopenia, or paraspinal muscle atrophy (PMA).

In some aspects, the myopathy is a primary myopathy. In one aspect, a primary myopathy comprises disuse atrophy resulting from, e.g., hip fracture, elective joint replacement, critical care myopathy, spinal cord injury or stroke. In some embodiments, the myopathy is a genetic muscle weakness associated with, for example, a muscular dystrophy.

In some aspects, the myopathy is a secondary myopathy, in which muscle loss or dysfunction is secondary to a disease pathology, such as denervation or cachexia, denervation associated with monitor neuron dysfunction. In some aspects, motor neuron dysfunction is due to genetic mutation(s) that affect motor neurons, such as, e.g., amyotrophic lateral sclerosis (ALS) and spinal muscular atrophy (SMA). In some embodiments, the secondary myopathy is a cachexia associated with renal failure, long-COVID, a cardiac condition, cancer or aging. In some aspects, the secondary myopathy is caused by a nerve injury, such as, nerve injury sustained during a medical procedure, such as surgeries. Detrimental effects of such injury to the function of a target tissue (e.g., target muscle) may be effectively treated by administration of the anti-myostatin antibodies or antigen binding fragments described herein. For example, such administration may prevent and/or alleviate myopathy, and/or facilitate recovery.

More particularly, the present invention provides bispecific antibodies and portions thereof, that specifically bind to myostatin with one binding portion, and to Activin A with the other binding portion. The antibodies bispecific described herein are valuable for the treatment, prevention, and/or mitigation of diseases or disorders linked to myostatin activity. In particular, these antibodies are beneficial in treating conditions that can be improved by enhancing muscle strength, power, mass, or function in an individual, or by positively influencing metabolic processes (e.g., carbohydrate, lipid, and protein metabolism) through the inhibition of myostatin activity. Examples of diseases, disorders, and conditions treatable with the anti-myostatin antibodies disclosed herein include, but are not limited to, sarcopenia, cachexia (whether idiopathic or secondary to conditions such as cancer, chronic kidney disease, or chronic obstructive pulmonary disease), muscle injuries, muscle wasting, and muscle atrophy, such as those arising from or associated with disuse, immobilization, bed rest, injury, medical treatments, or surgical interventions (e.g., hip fractures, hip or knee replacements) or the necessity for mechanical ventilation. The anti-myostatin antibodies may also be employed in the treatment, prevention, or mitigation of diseases including, but not limited to, cancer, obesity, diabetes, arthritis, multiple sclerosis, muscular dystrophy, amyotrophic lateral sclerosis, Parkinson's disease, osteoporosis, osteoarthritis, osteopenia, and metabolic syndromes (such as diabetes, obesity, nutritional disorders, organ atrophy, chronic obstructive pulmonary disease, and anorexia).

In some aspects, the bispecific antibodies, or antigen binding fragments thereof can be used in methods for treating or preventing muscle conditions and disorders, including paraspinal muscle atrophy, nerve injury-dependent muscle atrophy, postoperative nerve injury-dependent muscle atrophy, a lumbar spine surgery, a lumbar spine procedure (e.g., a lumbar fusion procedure, a lumbar nonfusion procedure, a posterior lumbar fusion procedure, an anterior lumbar fusion procedure, a minimally invasive (MIS) posterior lumbar decompression procedure, a minimally invasive (MIS) posterior lumbar fusion procedure, a non-MIS equivalent procedure), etc.

In one aspect, the methods of treatment with the bispecific inhibitors, e.g., antibodies, or antigen binding fragments thereof, described herein results in at least 1%, 2%, 3%, 4%, 5%, 6%, 7%, 8%, 9%, 10%, 12%, 15%, 18%, 20%, 25%, 30%, 40%, 50%, 60%, 70%, 75%, 80%, or 90% decrease in muscle atrophy. In one embodiment, methods of treatment with the myostatin inhibitors, e.g., antibodies, or antigen binding fragments thereof, described herein results in preventing at least a %, 2%, 3%, 4%, 5%, 6%, 7%, 8%, 9%, 10%, 12%, 15%, 18%, 20%, 25%, 30%, 40%, 50%, 60%, 70%, 75%, 80%, or 90% of the muscle atrophy.

The invention also provides bispecific antibodies and portions thereof, that specifically bind to activin A or portions of activin A, and methods for using such binding agents. The bispecific binding agents are useful to block or impair binding of human activin A and to myostatin.

By targeting Activin A, the bispecific binding antibodies or binding fragments thereof can be used to treat cancer, rheumatoid arthritis, chronic renal failure, congestive heart failure, and other conditions in which cachexia is a factor. The bispecific antibodies of the present invention can be used to reduce cachexia by targeting the activin A pathway. Activin A is the predominant form of activin. Diseases or conditions that can be treated with the novel bispecific antibodies and portions thereof include, e.g., muscle wasting and weakness, which are common in many disease states and conditions including aging, cancer cachexia, sepsis, denervation, disuse, inactivity, burns, HIV-acquired immunodeficiency syndrome (AIDS), chronic kidney or heart failure, unloading/microgravity, and muscular dystrophies. Activins are members of the TGF-beta superfamily. Activins function as stimulators and inhibitors, respectively, of pituitary follicle-stimulating hormone (FSH) secretion and biosynthesis.

The present invention provides compositions, kits, and methods relating to bispecific antibodies and binding fragments thereof, which bind to the activin A and myostatin, including those that agonize or antagonize activin A and myostatin, such as anti-activin A and myostatin antibodies, antibody fragments, and antibody derivatives, e.g., antagonistic anti-activin A and myostatin bispecific antibodies, bispecific antibody fragments, or bispecific antibody derivatives. Also provided are compositions, kits, and methods relating to molecules that specifically bind to activin A and myostatin.

In addition to promoting bone growth, the disclosure contemplates using the bispecific antibodies or binding fragments thereof as ActRIIb antagonists by blocking the interaction with Activin A in treating or preventing diseases or conditions that are associated with abnormal activity of an ActRIIb or an ActRIIb ligand. For example, ActRIIb antagonists may be used to treat human or animal disorders or conditions. Examples of such disorders or conditions of bone growth include, but are not limited to, metabolic disorders such as type 2 diabetes, impaired glucose tolerance, metabolic syndrome (e.g., syndrome X), and insulin resistance induced by trauma (e.g., burns or nitrogen imbalance); adipose tissue disorders (e.g., obesity); muscle and neuromuscular disorders such as muscular dystrophy (including Duchenne muscular dystrophy); amyotrophic lateral sclerosis (ALS); muscle atrophy; organ atrophy; frailty; carpal tunnel syndrome; congestive obstructive pulmonary disease; and sarcopenia, cachexia and other muscle wasting syndromes. Other examples include osteoporosis, especially in the elderly and/or postmenopausal women; glucocorticoid-induced osteoporosis; osteopenia; osteoarthritis; and osteoporosis-related fractures. Yet further examples include low bone mass due to chronic glucocorticoid therapy, premature gonadal failure, androgen suppression, vitamin D deficiency, secondary hyperparathyroidism, nutritional deficiencies, and anorexia nervosa.

Non-limiting examples of muscle-related diseases or conditions include neuromuscular disorders (e.g., muscular dystrophy and muscle atrophy), congestive obstructive pulmonary disease (and muscle wasting associated with COPD), muscle wasting syndrome, sarcopenia, cachexia, adipose tissue disorders (e.g., obesity), type 2 diabetes, and bone degenerative disease (e.g., osteoporosis). Other exemplary conditions include musculo-degenerative and neuromuscular disorders, tissue repair (e.g., wound healing), neurodegenerative diseases (e.g., amyotrophic lateral sclerosis), immunologic disorders (e.g., disorders related to abnormal proliferation or function of lymphocytes), and obesity or disorders related to abnormal proliferation of adipocytes.

Also provided are nucleic acids, and derivatives and fragments thereof, comprising a sequence of nucleotides that encodes all or a portion of a polypeptide that binds to activin A and to a polypeptide that binds myostatin, e.g., a nucleic acid encoding all or part of an anti-activin A antibody, antibody fragment, antibody variant, or antibody derivative, plasmids and vectors comprising such nucleic acids, and cells or cell lines comprising such nucleic acids and/or vectors and plasmids, and a nucleic acid encoding all or part of an anti-myostatin antibody, antibody fragment, antibody variant, or antibody derivative, plasmids and vectors comprising such nucleic acids, and cells or cell lines comprising such nucleic acids and/or vectors and plasmids, which are expressed together or separately for form the bispecific binding antibodies, antibody fragments, antibody variants, or antibody derivatives.

Also provided herein are methods that include, for example, methods of making, identifying, or isolating bispecific molecules that bind to activin A, such as anti-activin A antibodies, methods of determining whether a molecule binds to activin A and myostatin, methods of making compositions, such as pharmaceutical compositions, comprising a molecule that binds to activin A and myostatin, and methods for administering a molecule that binds activin A and myostatin to a subject, for example, methods for treating a condition mediated by activin A and myostatin, and for modulating a biological activity of activin A and myostatin in vivo or in vitro.

As used herein, the term “antibody” refers to an intact antibody or a binding fragment thereof that binds specifically to a target antigen. Binding fragments are produced by recombinant DNA techniques, or by enzymatic or chemical cleavage of intact antibodies. Binding fragments include Fab, Fab′, F(ab′)2, Fv, and single-chain variable fragment (scFv) antibodies. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay). The term “antibody” is used in the broadest sense, and specifically covers monoclonal antibodies (including full-length antibodies or other bivalent, Fc-region containing antibodies such as bivalent scFv Fc-fusion antibodies), polyclonal antibodies, multispecific antibodies (e.g., bispecific antibodies), and antibody fragments (e.g., Fab, Fab′, F(ab′)2, Fv, scFv) so long as they exhibit the desired biological activity. Antibodies (Abs) and immunoglobulins (Igs) are glycoproteins having the same structural characteristics. The present invention includes monoclonal antibodies (and binding fragments thereof) that are completely recombinant, in other words, where the complementarity determining regions (CDRs) are genetically spliced into a human antibody backbone, often referred to as veneering an antibody. Thus, in certain aspects, the monoclonal antibody is a fully synthesized antibody. In certain embodiments, the monoclonal antibodies (and binding fragments thereof) can be made in bacterial or eukaryotic cells, including plant cells.

As used herein, the term “antibody fragment” refers to a portion of a full-length antibody, generally the antigen-binding or variable region, and include Fab, Fab′, F(ab′)2, Fv, and scFv fragments. Papain digestion of antibodies produces two identical antigen-binding fragments, called the Fab fragment, each with a single antigen-binding site, and a residual “Fc” fragment, so-called for its ability to crystallize readily. Pepsin treatment yields an F(ab′)2 fragment that has two antigen-binding fragments which are capable of cross-linking antigen, and a residual other fragment (which is termed pFc′). As used herein, “functional fragment” with respect to antibodies, refers to Fv, F (ab) and F(ab′)2 fragments. As used herein, the “Fv” fragment is the minimum antibody fragment that contains a complete antigen recognition and binding site. This region consists of a dimer of one heavy and one light chain variable domain in a tight, non-covalent association (VH-VL dimer). It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The present invention includes bispecific or multispecific antibodies, or binding fragments thereof, which comprise at least a heavy chain variable region from the antibody family of the invention and may comprise a heavy and light chain variable region provided herein. Bispecific antibodies comprise at least the heavy chain variable region of an antibody specific for activin A and myostatin, and may comprise a heavy and light chain variable region. In some such embodiments, the second antibody specificity binds to a tumor associated antigen, a targeting antigen, e.g., integrins, etc., a pathogen antigen, a checkpoint protein, and the like. Various formats of bispecific antibodies are within the scope of the invention, including without limitation single chain polypeptides, two chain polypeptides, three chain polypeptides, four chain polypeptides, and multiples thereof.

The Fab fragment, also designated as F (ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)2 pepsin digestion product. Additional chemical couplings of antibody fragments are known to those of ordinary skill in the art.

Native antibodies and immunoglobulins are usually heterotetrameric glycoproteins of about 150,000 daltons, composed of two identical light (L) chains and two identical heavy (H) chains. Each light chain is linked to a heavy chain by at least one covalent disulfide bond, however, the number of disulfide linkages varies between the heavy chains of different immunoglobulin isotypes. Each heavy and light chain also has regularly spaced intrachain disulfide bridges. Each heavy chain has at one end a variable domain (VH) followed by the constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues form an interface between the light and heavy chain variable domains (Clothia et al., J. Mol. Biol. 186, 651-66, 1985); Novotny and Haber, Proc. Natl. Acad. Sci. USA 82 4592-4596 (1985), relevant portions incorporated herein by reference.

As used herein, an “isolated” antibody is one that has been identified and separated and/or recovered from a component of the environment in which it was produced. Contaminant components of its production environment are materials, which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequenator, such as at least 15 residues of sequence; or 3) to homogeneity by SDS-PAGE under reducing or non-reducing conditions using Coomasie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the terms “antibody mutant” or “antibody variant” refer to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, 91%, 92%, 93%, 94%, 95%, 96%, 97%, 98%, or 99%.

As used herein, the term “variable” in the context of the variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342:877), or both, that is Chothia plus Kabat. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al.) The constant domains are not involved directly in binding an antibody to its cognate antigen but exhibit various effector function, such as participation of the antibody in antibody-dependent cellular toxicity.

The light chains of antibodies (immunoglobulin) from any vertebrate species can be assigned to one of two clearly distinct types, called kappa and lambda, based on the amino sequences of their constant domain. Depending on the amino acid sequences of the constant domain of their heavy chains, “immunoglobulins” can be assigned to different classes. There are at least five (5) major classes of immunoglobulins: IgA, IgD, IgE, IgG, and IgM, and several of these may be further divided into subclasses (isotypes), e.g., IgG-1, IgG-2, IgG-3, and IgG4; IgA-1 and IgA-2. The subunit structures and three-dimensional configurations of different classes of immunoglobulins are well known.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed and claimed invention may be made by the hybridoma method first described by Kohler and Milstein, Nature 256, 495 (1975), relevant portions incorporated herein by reference.

All monoclonal antibodies used in accordance with the presently disclosed and claimed invention will be either (1) the result of a deliberate immunization protocol, as described in more detail hereinbelow; or (2) the result of an immune response that results in the production of antibodies naturally in the course of a disease or cancer.

As used herein, the term “binding affinity” refers to the strength of the sum total of noncovalent interactions between a single binding site of a molecule (e.g., an antibody or other binding molecule) and its binding partner (e.g., an antigen or receptor). The affinity of a molecule X for its partner Y can generally be represented by the dissociation constant (Kd). Affinity can be measured by common methods known in the art, including those described herein. Low-affinity antibodies bind antigen (or receptor) weakly and tend to dissociate readily, whereas high-affinity antibodies bind antigen (or receptor) more tightly and remain bound longer. In the case of antibodies, the antigen binding site binds to its cognate epitope.

As used herein, the terms “cancer,” “neoplasm,” and “tumor” are used interchangeably to refer to cells that exhibit autonomous, unregulated growth, such that they exhibit an aberrant growth phenotype characterized by a significant loss of control over cell proliferation. Cells of interest for detection, analysis, or treatment in the present application include precancerous (e.g., benign), malignant, pre-metastatic, metastatic, and non-metastatic cells. Cancers of virtually every tissue are known. As used herein, the phrase “cancer burden” refers to the number of cancer cells or cancer volume in a subject. Reducing a cancer burden refers to reducing the number of cancer cells or the cancer volume in a subject. As used herein, the term “cancer cell” refers to any cell that is a cancer cell or is derived from a cancer cell, e.g., a clone of a cancer cell. Many types of cancers are known to those of skill in the art, including solid tumors such as carcinomas, sarcomas, glioblastomas, melanomas, lymphomas, myelomas, etc., and circulating cancers such as leukemias, including specifically B cell leukemias, T cell leukemias, etc. Examples of cancer that may be treated with the bispecific, modulatable antibody or fragments thereof include but are not limited to, ovarian cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

The uses of the monoclonal antibodies of the presently disclosed and claimed invention may require administration of such or similar monoclonal antibody to a subject, such as a human. However, when the monoclonal antibodies are produced in a non-human animal, such as a rodent or chicken, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed and claimed invention can be “humanized”, that is, the antibodies are engineered such that antigenic portions thereof are removed and like portions of a human antibody are substituted therefore, while the antibodies' affinity for the activin A and myostatin and the target antigen is retained. This engineering may only involve a few amino acids, or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, relevant portions incorporated herein by reference.

Humanized forms of antibodies are chimeric immunoglobulins, immunoglobulin chains or fragments thereof (such as Fab, Fab′, F(ab′)2, Fv, scFv or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988; Verhoeyen et al., 1988), by substituting nonhuman (i.e., rodent, chicken) CDRs or CDR sequences for the corresponding sequences of a human antibody, see, e.g., U.S. Pat. No. 5,225,539. In some instances, Fv framework residues of the human immunoglobulin are replaced by corresponding non-human residues from the donor antibody. Humanized antibodies can also comprise residues that are found neither in the recipient antibody nor in the imported CDR or framework sequences. In general, the humanized antibody will comprise substantially all of, at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin.

As used herein, the term “chimeric” antibody refers to a portion of the heavy and/or light chain that is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. A chimeric antibody will have two distinct antigen binding sites, which may be against the same or different antigens.

Antibody fragments of the disclosure retain activin A and myostatin antigen binding specificity and binding to the target antigen. Antibody fragments include antigen-binding fragments (Fab), variable fragments (Fv) containing VH and VL sequences, single chain variable fragments (scFv) containing VH and VL sequences linked together in one chain, single chain antibody fragments (scAb) or other antibody variable region fragments, such as retaining antigen binding specificity.

As used herein, a “subject” may be a mammalian subject. Mammalian subjects include, humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion animal (e.g., cats, dogs).

The presently disclosed and claimed invention further includes the use of fully human monoclonal antibodies cross-reactive against activin A and myostatin. Fully human antibodies essentially relate to antibody molecules in which the entire sequence of both the light chain and the heavy chain, including the CDRs, arise from human genes. Such antibodies are termed “human antibodies” or “fully human antibodies” herein. Human monoclonal antibodies can be prepared by, e.g., the trioma technique; the human B-cell hybridoma technique (see Kozbor, et al., Hybridoma, 2:7 (1983)) and the EBV hybridoma technique to produce human monoclonal antibodies (see Cole, et al., PNAS 82:859 (1985)), or as taught herein. Human monoclonal antibodies may be utilized in the practice of the presently disclosed and claimed invention and may be produced by using human hybridomas (see Cote, et al., PNAS 80:2026 (1983)) or by transforming human B-cells with Epstein Barr Virus in vitro (see Cole, et al., 1985), relevant portions incorporated herein by reference.

In addition, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example but not by way of limitation, in U.S. Pat. Nos. 5,545,807; 5,545,806; 5,569,825; 5,625,126; 5,633,425; 5,661,016, and in Marks et al., J Biol. Chem. 267:16007, (1992); Lonberg et al., Nature, 368:856 (1994); Morrison, 1994; Fishwild et al., Nature Biotechnol. 14:845 (1996); Neuberger, Nat. Biotechnol. 14:826 (1996); and Lonberg and Huszar, Int Rev Immunol. 13:65 (1995), relevant portions incorporated herein by reference.

A method for producing an antibody of interest, such as a human antibody, is disclosed in U.S. Pat. No. 5,916,771, issued to Hori et al. on Jun. 29, 1999, and incorporated herein by reference. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

Non-amino acid cleavage polymers or linkers can include, e.g., nucleic acids, lipids, carbohydrates, or chemical linkers, such as those that are cleaved or dissociated by, e.g., light, radiation, electromagnetic, pH, chemically, or enzymatically.

Structural-switch linkers can include amino acid or non-amino acid polymers that adopt an inactivated prodrug conformation with the mask blocking the antibody-antigen binding site. Upon exposure to specific microenvironment conditions such as pH, enzyme(s), or metabolite(s)—the linker switches to the activated antibody conformation where the mask no longer blocks antibody-antigen binding.

As used herein, the term “vector” refers to a nucleic acid molecule capable of transporting another nucleic acid to which it has been linked. One type of vector is a “plasmid”, which refers to a circular double-stranded DNA into which additional DNA segments are inserted. Another type of vector is a viral vector in which DNA segments are inserted into a viral genome. Certain vectors are capable of autonomous replication in a host cell into which they are introduced (e.g., bacterial vectors having a bacterial and an episomal origin of replication, or non-episomal mammalian vectors can be integrated into the genome of a host cell upon introduction into the host cell, and thereby are replicated along with the host genome. Certain vectors are capable of directing the expression of genes to which they are operably linked. Such vectors are referred to herein as “recombinant expression vectors” (or simply, “recombinant vectors”). Generally, expression vectors useful for expression in mammalian cells using recombinant DNA techniques are often in the form of plasmids.

As used herein, the term “host cell” or “recombinant host cell” refer to a cell that has been genetically altered, or is capable of being genetically altered by introduction of an exogenous polynucleotide, such as a recombinant plasmid or vector. A host cell refers not only to a particular subject cell but also to the progeny of such a host cell. Because certain modifications may occur in succeeding generations due to either mutation or environmental influences, such progeny may not, in fact, be identical to the parent cell, but are still included within the scope of the term host cell.

As used herein, the term “treatment” refers to both therapeutic treatment and prophylactic or preventative measures. Those in need of treatment include those already with the disease or disorder as well as those in which the disorder is to be prevented. The effect may be prophylactic in terms of completely or partially preventing a disease or symptom thereof and/or may be therapeutic in terms of a partial or complete cure for a disease and/or adverse effects attributable to the disease. A “treatment” refers to any treatment of a disease or disorder in a mammal and includes: (a) preventing the disease from occurring in a subject that may be predisposed to the disease but has not yet been diagnosed as having it; (b) relieving the disease, i.e., causing regression of the disease; or (c) inhibiting the disease, i.e., arresting its development. The therapeutic agent may be administered before, during, or after the onset of disease or injury. The treatment of ongoing disease, where the treatment stabilizes or reduces the undesirable clinical symptoms of the patient, is of particular interest. Such treatment is desirably performed prior to the complete loss of function in the affected tissues. The subject therapy may be administered during the symptomatic stage of the disease, and in some cases after the symptomatic stage of the disease. Treatment may be delivered prophylactically (prior to showing signs of an autoimmune disease or cancer) or therapeutically (following the presence of an autoimmune disease or cancer). In one aspect, the invention also includes using a first bispecific antibody or antigen binding fragment having a predetermined activity is used during a first phase of treatment, and a second bispecific antibody or antigen binding fragment having a different predetermined activity is used during a second phase of treatment, and optionally using one or more different bispecific antibody or antigen binding fragment having a predetermined activity is used during a first phase of treatment for any additional phases of treatment. In other words, a first bispecific antibody or antigen binding fragment having a first predetermined activity is used during a first phase of treatment, and a second bispecific antibody or antigen binding fragment having a second predetermined activity is used during a second phase of treatment, and optionally using one or more different bispecific antibody or antigen binding fragment having a different predetermined activity is used during a first phase of treatment for any additional phases of treatment.

As used herein, the term “disorder” refers to any condition that would benefit from treatment with the bispecific, modulatable antibody or fragments thereof. This includes chronic and acute disorders or diseases including those infectious or pathological conditions that predispose the mammal to the disorder in question.

An antibody or antibody fragment can be generated with an engineered sequence or

glycosylation state to confer preferred levels of activity in antibody-dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

Alternatively, or additionally, it may be useful to combine amino acid modifications with one or more further amino acid modifications that alter complement component Clq binding and/or the complement-dependent cytotoxicity (CDC) function of the Fc region of an IL-23p19 binding molecule. The binding polypeptide of particular interest may be one that binds to Clq and displays complement-dependent cytotoxicity. Polypeptides with pre-existing Clq binding activity, optionally further having the ability to mediate CDC may be modified such that one or both of these activities are enhanced. Amino acid modifications that alter Clq and/or modify its complement-dependent cytotoxicity function are described, for example, in W0/0042072, which is hereby incorporated by reference.

An Fc region of an antibody can be designed to alter the effector function, e.g., by modifying Clq binding and/or FcγR binding and thereby changing complement-dependent cytotoxicity (CDC) activity and/or antibody-dependent cell-mediated cytotoxicity (ADCC) activity. These “effector functions” are responsible for activating or diminishing a biological activity (e.g., in a subject). Examples of effector functions include, but are not limited to: Clq binding; CDC; Fc receptor binding; ADCC; phagocytosis; down-regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

The Fc region can be a wild-type Fc region, a mutated Fc region, a monomeric wild-type Fc region, a monomeric mutant Fc region, a dimeric wild-type Fc region, or a dimeric mutant Fc region, a second variable heavy region and a second Fc region, or a second variable heavy region and a second Fc region, and may further include an uncleavable flexible linker and a second variable light region and a second heavy variable region, or a second Fc region and an uncleavable flexible linker and payload, such as a toxin, a cytokine, an anti-cancer agent, or another antibody.

Non-limiting examples of payload include, e.g., cytotoxic proteins or peptides include a bacterial cytotoxin such as an alpha-pore forming toxin (e.g., cytolysin A from E. coli), a beta-pore-forming toxin (e.g., α-Hemolysin, PVL-panton Valentine leukocidin, aerolysin, clostridial Epsilon-toxin, Clostridium perfringens enterotoxin), binary toxins (anthrax toxin, edema toxin, C. botulinum C2 toxin, C. spirofome toxin, C. perfringens iota toxin, C. difficile cyto-lethal toxins (A and B)), prion, parasporin, a cholesterol-dependent cytolysins (e.g., pneumolysin), a small pore-forming toxin (e.g., Gramicidin A), a cyanotoxin (e.g., microcystins, nodularins), a hemotoxin, a neurotoxin (e.g., botulinum neurotoxin), a cytotoxin, cholera toxin, diphtheria toxin, Pseudomonas exotoxin A, tetanus toxin, or an immunotoxin (idarubicin, ricin A, CRM9, Pokeweed antiviral protein, DT). Exemplary apoptotic triggering proteins or peptides include apoptotic protease activating factor-1 (Apaf-1), cytochrome-c, caspase initiator proteins (CASP2, CASP8, CASP9, CASP10), apoptosis inducing factor (AIF), p53, p73, p63, Bcl-2, Bax, granzyme B, poly-ADP ribose polymerase (PARP), and P 21-activated kinase 2 (PAK2).

The payload can also be a cytotoxic agent, such as doxorubicin (adriamycin), gemcitabine (gemzar), daunorubicin, procarbazine, mitomycin, cytarabine, etoposide, methotrexate, venorelbine, 5-fluorouracil (5-FU), vinca alkaloids such as vinblastine or vincristine; bleomycin, paclitaxel (taxol), docetaxel (taxotere), aldesleukin, asparaginase, busulfan, carboplatin, cladribine, camptothecin, CPT-11, 1O-hydroxy-7-ethylcamptothecin (SN38), dacarbazine, S-I capecitabine, ftorafur, 5′deoxyfluorouridine, UFT, eniluracil, deoxycytidine, 5-azacytosine, 5-azadeoxycytosine, allopurinol, 2-chloroadenosine, trimetrexate, aminopterin, methylene-10-deazaminopterin (MDAM), oxaplatin, picoplatin, tetraplatin, satraplatin, platinum-DACH, ormaplatin, CI-973, JM-216, and analogs thereof, epirubicin, etoposide phosphate, 9-aminocamptothecin, 10,11-methylenedioxycamptothecin, karenitecin, 9-nitrocamptothecin, TAS 103, vindesine, L-phenylalanine mustard, ifosphamidemefosphamide, perfosfamide, trophosphamide carmustine, semustine, epothilones A-E, tomudex, 6-mercaptopurine, 6-thioguanine, amsacrine, etoposide phosphate, karenitecin, acyclovir, valacyclovir, ganciclovir, amantadine, rimantadine, lamivudine, zidovudine, bevacizumab, trastuzumab, rituximab, 5-Fluorouracil, and combinations thereof.

Non-limiting examples of cytokines include: growth hormone; parathyroid hormone; thyroxine; insulin; proinsulin; relaxin; prorelaxin; glycoprotein hormones; hepatic growth factor; fibroblast growth factor; prolactin; placental lactogen; TNF-α; mullerian-inhibiting substance; gonadotropin-associated peptide; inhibin; activin; vascular endothelial growth factor; integrin; thrombopoietin (TPO); nerve growth factors; platelet-growth factor; placental growth factor, transforming growth factors (TGFs); insulin-like growth factor-1 and -11; erythropoietin (EPO); osteoinductive factors; interferons; colony stimulating factors (CSFs); lymphotoxin-alpha; lymphotoxin-beta; CD27L; CD30L; FASL; 4-1 BBL; OX40L; TRAIL; IL-1; IL-2; IL-3; IL-4; IL-5; IL-6; IL-7; IL-8; IL-9; IL-10; IL-11; IL-12; IL-13; IL-15; IL-18; IL-21; IL-22; IL-23; IL-33; IFN-α; IFN-b; IFN-g; IFN-g inducing factor (IGIF); bone morphogenetic protein (BMP); leukemia inhibitory factor (LIF); or kit ligand (KL).

Non-limiting examples of anti-cancer drug include, e.g., dihydroxyvitamin D3, 4-ipomeanol, 5-ethynyluracil, 9-dihydrotaxol, abiraterone, acivicin, aclarubicin, acodazole hydrochloride, acronine, acylfiilvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DLPTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisazuidinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caraceraide, carbetimer, carboplatin, carboxamide-amino-triazole, carboxyamidotriazole, carest M3, carmustine, earn 700, cartilage derived inhibitor, carubicin hydrochloride, carzelesin, casein kinase inhibitors, castanosperrnine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethyhiorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocannycin SA, ebselen, ecomustine, edatrexate, edelfosine, edrecolomab, eflomithine, eflomithine hydrochloride, elemene, elsarnitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, fluorocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ihnofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatm, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide, lobaplatin, lombricine, lometrexol, lometrexol sodium, lomustine, lonidamine, losoxantrone, losoxantrone hydrochloride, lovastatin, loxoribine, lurtotecan, lutetium texaphyrin lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, 06-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazorurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RH retinamide, RNAi, rogletimide, rohitukine, romurtide, roquinimex, rubiginone Bl, ruboxyl, safingol, safingol hydrochloride, saintopin, sarcnu, sarcophytol A, sargramostim, SDII mimetics, semustine, senescence derived inhibitor 1, sense oligonucleotides, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosafe sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine or vinorelbine tartrate, vinrosidine sulfate, vinxaltine, vinzolidine sulfate, vitaxin, vorozole, zanoterone, zeniplatin, zilascorb, zinostatin, zinostatin stimalamer, or zorubicin hydrochloride.

For example, one can generate a variant Fc region of an antibody with improved Clq binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen-binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine, and glycine. However, other residues can function as well. Phage display can be used to rapidly select tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×106 different members) is displayed on filamentous phage and subjected to affinity selection with hapten. The population of selected variants exhibited significant increases in binding activity but retained considerable sequence diversity. Sequence analysis revealed a conserved proline in the linker two residues after the VH C terminus and an abundance of arginines and prolines at other positions as the only common features of the selected tethers. In certain embodiments, the antibody fragments are further modified to increase their serum half-life by using modified Fc regions or mutations to the various constant regions, as are known in the art.

In certain embodiments, the antibodies of the present invention are formulated for administration to humans. For example, the antibodies of the present invention can be included in a pharmaceutical composition formulated for an administration that is: intranasal, intrapulmonary, intrabronchial, intravenous, oral, intraadiposal, intraarterial, intraarticular, intracranial, intradermal, intralesional, intramuscular, intrapericardial, intraperitoneal, intrapleural, intravesicular, local, mucosal, parenteral, enteral, subcutaneous, sublingual, topical, transbuccal, transdermal, via inhalation, via injection, in creams, in lipid compositions, via a catheter, via a lavage, via continuous infusion, via infusion, via local delivery, or via localized perfusion, and wherein the composition is a serum, drop, gel, ointment, spray, reservoir, or mist.

As used herein, the term “antigen” refers to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides, which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts, which produce the antigens.

As used herein, the term “target antigens” refers to a refers to a molecule containing one or more epitopes (either linear, conformational or both) against which an antibody or binding fragment thereof has been raised and will be combined with the activin A and myostatin antibodies or binding fragments thereof what will be combined to form a bispecific antibody that has a predetermined activity.

As used herein, the terms “predetermined activity” or “predetermined immune cell activity” or “predetermined threshold” refer to an activity of the bispecific or multivalent antibody or antigen binding fragments thereof that cause binding of the bispecific or multivalent antibody or antigen binding fragments thereof to one or more cognate binding targets and that together trigger an immune response, which can include, e.g., T cell killing, CD8 T cell activation, T cell proliferation, B cell proliferation, cytokine secretion, lymphokine secretion, TNF-alpha secretion, IFN-gamma secretion, IL-2 secretion, IL-10 secretion, antigen binding, CD3 binding, T cell anergy, T cell tolerance, NK cell activation, antibody dependent cell cytotoxicity (ADCC), T cell dependent cell cytotoxicity (TDCC), or immune cell suppression, secretion of immune activating cytokine(s)/lymphokine(s), secretion of immune suppressing cytokine(s)/lymphokine(s), T effector activity, or T regulatory cell activity. In another example, the predetermined phenotype, function or attribute is an effector function associated with T cell activation state, is a cell surface phenotype or is a pharmacokinetic activity. Non-limiting examples of phenotype activities are the expression or surface expression of one or more markers generally associated with one or more sub-types or subpopulations of T cells, or phenotypes thereof. T cell subtypes and subpopulations may include CD4+ and/or of CD8+ T cells and subtypes thereof that may include naive T cells, effector T cells, memory T cells and sub-types thereof, such as stem cell memory T, central memory T, effector memory T, T effector memory cells or terminally differentiated effector memory T cells, tumor-infiltrating lymphocytes (TIL), immature T cells, mature T cells, helper T cells, cytotoxic T cells, mucosa-associated invariant T (MAIT) cells, regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

As used herein, the term “epitope” refers to a specific amino acid sequence or molecule (such as a carbohydrate, small molecule, lipid, etc.) that when present in the proper conformation, provides a reactive site for an antibody (e.g., B cell epitope) or in the case of a peptide to a T cell receptor (e.g., T cell epitope).

Portions of a given polypeptide that include a B-cell epitope can be identified using any number of epitope mapping techniques that are known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad Sci. USA 81:3998-4002; Geysen et al. (1986) Molec. Immunol. 23:709-715.

As used herein, the term “substantially purified” refers to isolation of a substance (compound, polynucleotide, protein, polypeptide, polypeptide composition) such that the substance comprises the majority percent of the sample in which it resides. Typically, in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

The practice of the present invention employs, unless otherwise indicated, conventional methods of chemistry, biochemistry, molecular biology, immunology, and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York), relevant portion incorporated herein by reference.

Conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

The skilled artisan will recognize that antibodies that exhibit little or no binding to a target antigen can be described as having a low affinity, and a high equilibrium dissociation constant (KD) for the target antigen. The skilled artisan will also recognize that antibodies that exhibit little or no binding to a collective assembly of target antigenic epitopes can be described as having a low avidity, and a high equilibrium dissociation constant (KD) for the collective assembly of target antigenic epitopes.

In some embodiments, provided herein are bispecific, modulatable antibody or fragments thereof having a binding affinity (KD) to activin A and/or myostatin of about 5 μM to about 5 pM, about 1 μM to about 5 pM, about 0.5 μM to about 5 pM, about 0.1 μM to about 5 pM, about 50 nM to about 5 pM, about 10 nM to about 5 pM, about 5 nM to about 5 pM, about 1 nM to about 5 pM, about 0.5 nM to about 5 pM, about 0.1 nM to about 5 pM, about 50 pM to about 5 pM, about 10 pM to about 5 pM.

In some embodiments, bispecific, modulatable antibody or fragments thereof have a binding avidity (KD) to activin A and/or myostatin of about 500 nM to about 0.1 pM, about 100 nM to about 0.1 pM, about 50 nM to about 0.1 pM, about 10 nM to about 0.1 pM, about 5 nM to about 0.1 pM, about 1 nM to about 0.1 pM, about 0.5 nM to about 0.1 pM, about 0.1 nM to about 0.1 pM, about 50 pM to about 0.1 pM, about 10 pM to about 0.1 pM, about 5 pM to about 0.1 pM, about 1 pM to about 0.1 pM, about 0.5 pM to about 0.1 pM.

In some embodiments, bispecific, modulatable antibody or fragments thereof have a half maximal effective concentration (EC50) to anti-activin A and/or myostatin of about 500 nM to about 0.001 nM, about 100 nM to about 0.001 nM, about 50 nM to about 0.001 nM, about 10 nM to about 0.001 nM, about 5 nM to about 0.001 nM, about 1 nM to about 0.001 nM, about 0.5 nM to about 0.001 nM, about 0.1 nM to about 0.001 nM, about 0.05 nM to about 0.001 nM, about 0.01 nM to about 0.001 nM, about 0.005 nM to about 0.001 nM.

In some embodiments, the bispecific, modulatable antibody or fragments thereof is a full-length antibody (referring to an antibody with two heavy and two light chains attached to the Fc domain, giving a ‘Y’ shape). In some embodiments the Fc domain (or simply referred to as an Fc) is a human Fc domain. In some embodiments, the Fc domain of an anti-CD3 antibody is from a human IgG1, human IgG2, human IgG3, or human IgG4.

Provided herein are sequences for exemplary anti-activin A and/or myostatin bispecific antibodies and antigen binding fragments thereof that are used in combination with an antigen binding fragment that targets an antigen to form a bispecific antibody, scFV, or fragments thereof. Included are complementarity determining region (CDR) sequences and the variable heavy and light domain sequences (VH, VL) that constitute the activin A and/or myostatin antigen binding domains of the disclosure. The discovery of these antibodies is detailed in the Examples section.

Provided herein are sequences for exemplary target antigen binding antibodies and antigen binding fragments thereof that are used in combination with the anti-activin A and/or myostatin antibody or antigen binding fragment thereof to form the bispecific antibody, scFV, or fragments thereof. Included are complementarity determining region (CDR) sequences and the variable heavy and light domain sequences (VH, VL) that constitute the activin A and/or myostatin antigen binding domains of the disclosure. The discovery of these antibodies is detailed in the Examples section.

As referred below, a light chain variable (VL) domain CDR1 region is referred to as CDR-L1; a VL CDR2 region is referred to as CDR-L2; a VL CDR3 region is referred to as CDR-L3; a heavy chain variable (VH) domain CDR1 region is referred to as CDR-H1; a VH CDR2 region is referred to as CDR-H2; and a VH CDR3 region is referred to as CDR-H3. The table herein provide exemplary CDR combinations of antibodies of the disclosure.

Each antibody arm may be connected by a linker, e.g., a flexible linker. An exemplary linker comprises the following amino acid sequence: GGGGS (SEQ ID NO:33), GGGGG (SEQ ID NO:34), or even GGGGSGGGGSGGGGS (SEQ ID NO:35).

Uses of bispecific, modulatable antibody or fragments thereof. Therapeutic bispecific, modulatable antibody or fragments thereof.

In some embodiments, the bispecific, modulatable antibody or fragments thereof provided herein are useful for the treatment of a disease or condition involving an immune response. In some embodiments, the bispecific, modulatable antibody or fragments thereof provided herein are useful for the treatment of an autoimmune disease. An autoimmune disease is generally a harmful immune response to a self-antigen. Examples of autoimmune diseases include, e.g., alopecia, ankylosing spondylitis, atopic dermatitis, celiac disease, Crohn's disease, cutaneous lupus erythematosus (CLE), lupus nephritis, multiple sclerosis, neuromyelitis optica, psoriasis, psoriatic arthritis, rheumatoid arthritis, scleroderma, Sjogren's syndrome, systemic lupus, systemic lupus erythematosus (SLE), temporal arteritis, type I diabetes, ulcerative colitis, uveitis, and vitiligo.

In some embodiments, the bispecific, modulatable antibody or fragments thereof provided herein are useful for the treatment of a hyperinflammatory disease. A hyperinflammatory disease consists of a potentially harmful overstimulated immune response. Examples of hyperinflammatory diseases include, e.g., chronic allergy, hypersensitivity vasculitis, and T cell hypersensitivity disease.

In other embodiments, the bispecific, modulatable antibody or fragments thereof provided herein are useful for the treatment of cancer. In certain aspect, the bispecific, modulatable antibody or fragments thereof are used alone or in combination to target the cancer cells. In other example, the bispecific, modulatable antibody or fragments thereof are used alone or in combination to target regulatory T cells that are reducing or blocking an immune response to the cancer cells.

Administration of Therapeutic bispecific, modulatable antibody or fragments thereof.

The in vivo administration of the therapeutic bispecific, modulatable antibody or fragments thereof described herein may be carried out intravenously, intramuscularly, subcutaneously, topically, orally, transdermally, subdermally, intraperitoneally, intraorbitally, intrathecally, intraventricularly, intranasally, transmucosally, through implantation, or through inhalation. Intravenous administration may be carried out via injection or infusion. In some embodiments, the bispecific, modulatable antibody or fragments thereof of the disclosure are administered intravenously. In some embodiments, the bispecific, modulatable antibody or fragments thereof of the disclosure are administered subcutaneously. Administration of the therapeutic bispecific, modulatable antibody or fragments thereof may be performed with any suitable excipients, carriers, or other agents to provide suitable or improved tolerance, transfer, delivery, and the like.

### EXAMPLES

### Antibody Expression and Purification.

Antibody expression plasmids were transiently introduced into an animal cell line using the ExpiFectamine CHO Transfection Kit (ThermoFisher; Cat #A29129) to yield transfectants that produced antibody. For a host cell line, ExpiCHO-S (ThermoFisher; Cat #A29127) was used. After 6-12 days of growth post introduction of DNA, cell suspensions of ExpiCHO were harvested via centrifugation for 20 min at 4,000×g, and then filtered using 0.2 μm Disposable PES Filter units (FisherScientific, Cat #FB12566504). Antibody was recovered from filtrate using Protein A purification (HiTrap MabSelect SuRe; Cytiva Cat #GE11-0034-93).

### HEK293SBE SMAD Signaling Assay.

HEK293 cells stably expressing a luciferase reporter gene linked to a SMAD binding element promoter (SBE) (BPS Bioscience, Cat #60653) were cultured in MEM media (Cytiva, Cat #SH30024.01) supplemented with 10% FBS (MilliporeSigma, Cat #F4135), 1% non-essential amino acids (Gibco, Cat #11140-050), 1 mM Na pyruvate (Gibco, Cat #11360-070), 1% Penicillin/Streptomycin (Corning, Cat #30-002-CI), and 400 μg/mL of Geneticin (Gibco, Cat #10131035) according to manufacturer's instructions.

To determine SMAD 2/3 activation, the HEK293/SBE/Luc cells were seeded in white, clear bottom 384-well plate (Greiner Bio-One, Cat #781098) at 3000 cells/well using Assay Media consisting of MEM media supplemented with 0.5% FBS (Gibco, Cat #26400044), 1% non-essential amino acids, 1 mM Na pyruvate, and 1% Penicillin/Streptomycin. 18-24 hours following plating, the cells were treated with the indicated antibodies at final dose range of 0.001-66.67 nM after one hour, 37° C./5% CO₂, antibody pre-incubation with either Myostatin (PeproTech, Cat #120-00) or Activin A (PeproTech, Cat #120-14E) or both, at a final concentration of 5 nM for Myostatin and 1 nM for Activin A. Following 18 hours of antibody/ligand incubation, 1 volume of ONE-Step Luciferase reagent (BPS Bioscience, Cat #60690) was added to cells and plates were shaken for 15 minutes at room temperature to facilitate lysis. After 15 minutes, luminescence was read on a SpectraMax ID5 (Molecular Devices) with 1000 ms integration time. GraphPad Prism 10.2.2 was used to calculate IC₅₀ values, generate graphs, and perform statistical analysis.

### Myoblast Differentiation Assay.

Primary human myoblast cells were purchased from Cook Myocite (Cat #SK-1111-P01457-24M). The cells were cultured in SkGM-2 Skeletal Muscle Cell Growth Media (Lonza, Cat #CC-3246), supplemented with the Medium-2 BulletKit (Lonza, Cat #CC-3244). Cell passage 3rd-6^{th} were used for this experiment.

To evaluate the effectiveness of anti-myostatin/activin A bispecific antibodies in rescuing myoblast differentiation attenuated by myostatin, activin A, GDF11, or the mixture of GDF8/GDF11/Activin A, cells were seeded at 10,000 cells per well in a 96-well plate (Corning, high throughput content imaging, cat #4680) and incubated overnight in a cell culture incubator. Cell samples were then treated with the indicated antibodies at a final dose range of 0.0017-200 nM after a one-hour pre-incubation at room temperature with either Myostatin (PeproTech, Cat #120-00), GDF-11 (PeproTech, Cat #120-11), Activin A, or the mixture of GDF8/GDF11/Activin A at a final concentration of 10 nM for GDF8 and GFF11 and 3 nM for Activin A. Cell samples were incubated in a cell culture incubator at 37° C. with 5% CO2 for 72 hours. After incubation, the samples were washed twice with 1×PBS and then fixed with 4% paraformaldehyde (Thermo Scientific, Cat #J19943.K2) for 30 minutes at room temperature. The samples were then washed twice with 1×PBS and blocked with blocking buffer (2% FBS, 0.2% Triton X-100 in 1×PBS) for 1 hour at room temperature. Prepare the anti-MF20 antibody (eBioscience, Cat #53-6503-82) by diluting the stock antibody 1:1000 and DAPI 1:10000 in antibody dilution buffer (2% FBS, 0.01% Triton X-100 in 1×PBS). Remove the blocking buffer and add 25 μL/well of the antibody solution. Incubate the samples for 1 hour at room temperature. Remove the antibody solution and wash the samples twice with 1×PBS. The samples are now ready for imaging. Acquire images of the samples using a 20× objective on the ImageExpress (Molecular Devices). Analyze the samples using custom analysis with ImageExpress software. The myoblast differentiation was evaluated by myotube coverage and fusion index. GraphPad Prism 10.2.2 was used to calculate EC50 values and generate graphs.

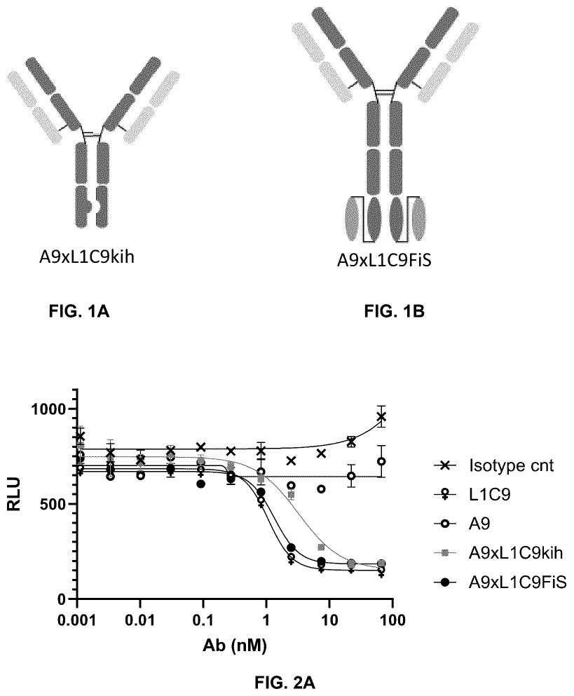

FIGS. 1A and 1B are representations of two examples of the bispecific antibody constructs of the present disclosure using that anti-activin A antibody A9 and the anti-myostatin antibody L1C9 binding domains. FIG. 1A shows a bifunctional A9×L1C9kih that includes Knob in Hole Fc domains. FIG. 1B shows a bifunctional A9×L1C9FiS that is an IgG-ScFv.

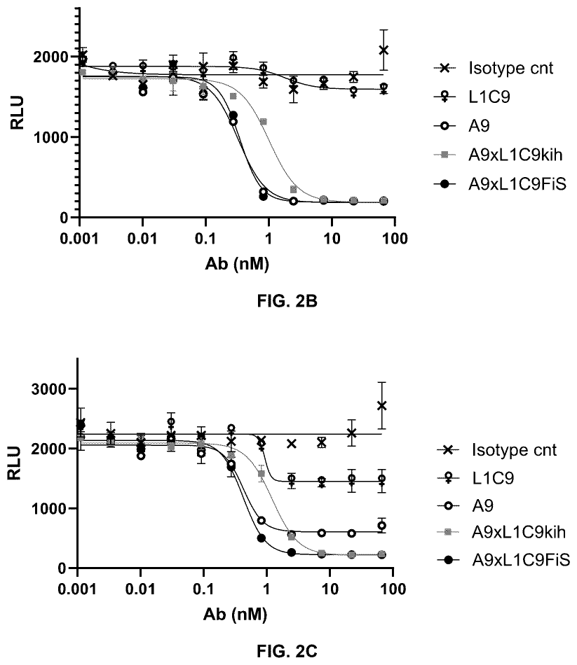

FIGS. 2A to 2C are graphs comparing the activity of the anti-activin A antibody A9 and the anti-myostatin antibody L1C9 binding domains and the bifunctional constructs. FIG. 2A: Reporter cell line to measure GDF8 antagonism. FIG. 2B: Reporter cell line to measure Activin A antagonism. FIG. 2C: Reporter cell line to measure combined GDF8 and Activin A antagonism.

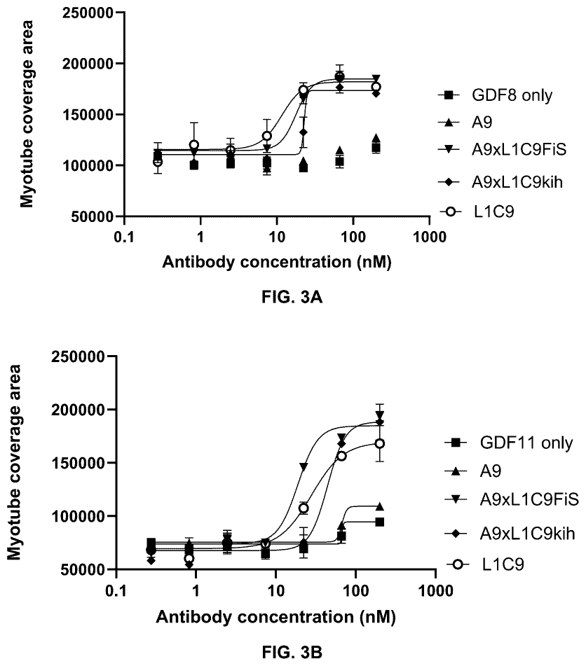

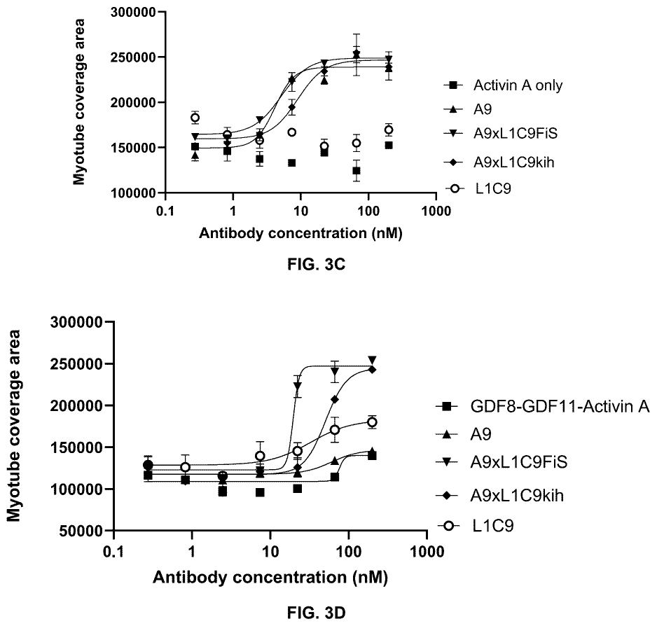

FIG. 3A to 3D are graphs comparing the activity of the anti-activin A antibody A9 and the anti-myostatin antibody L1C9 binding domains and the bifunctional constructs. FIG. 3A: Myotube coverage as measure of myoblast differentiation. GDF8 antagonism. FIG. 3B: Myotube coverage as measure of myoblast differentiation. GDF11 antagonism. FIG. 3C: Myotube coverage as measure of myoblast differentiation. Activin A antagonism. FIG. 3D: Myotube coverage as measure of myoblast differentiation. Combined GDF8, GDF11, and Activin A antagonism.

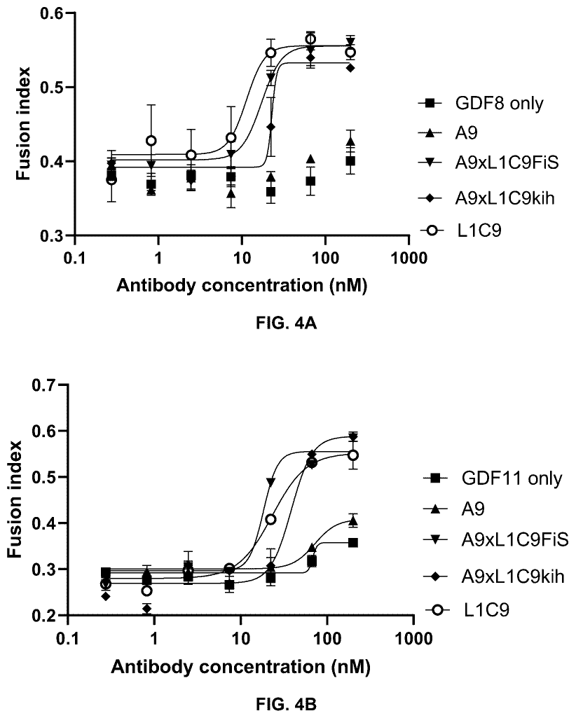

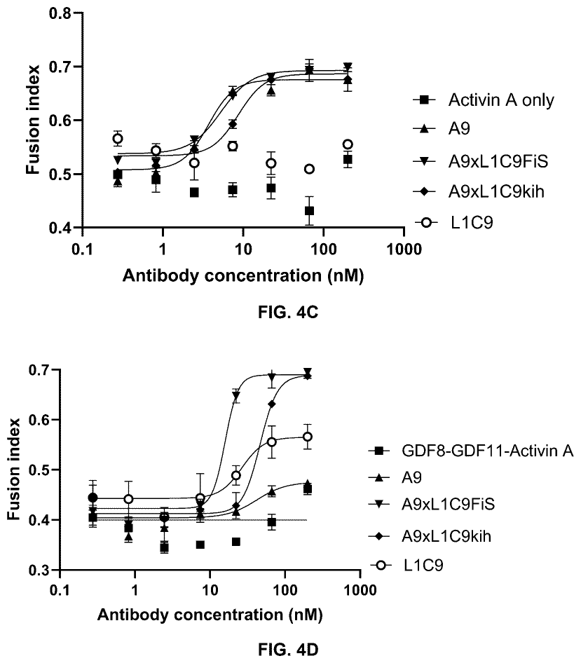

FIGS. 4A to 4D are graphs comparing the activity of the anti-activin A antibody A9 and the anti-myostatin antibody L1C9 binding domains and the bifunctional constructs. FIG. 4A: Fusion index as measure of myoblast differentiation. GDF8 antagonism. FIG. 4B: Fusion index as measure of myoblast differentiation. GDF11 antagonism. FIG. 4C: Fusion index as measure of myoblast differentiation. Activin A antagonism. FIG. 4D: Fusion index as measure of myoblast differentiation. Combined GDF8, GDF11, and Activin A antagonism.

TABLE 1
Anti-myostatin and anti-Activin A Heavy and Light chain
amino and nucleic acids

| Name | VH | VL | hCDR1 | hCDR2 | hCDR3 | 1CDR1 | 1CDR2 | 1CDR3 |
|---|---|---|---|---|---|---|---|---|
| LIC9 | EVQLVE | EIVLTQSP | GLTFS | ITSSGG | ARLP | SSVS | ATS | QHFS |
| Anti- | SGGGLV | GTLSLSP | RYP | ST | DY | SSY | | GYHF |
| myo- | QPGGSL | GERATLS | | | | | | T |
| statin | RLSCAA | CRASSSV | | | | | | |
| | SGLTFSR | SSSYLHW | | | | | | |
| | YPMSW | YQQKPG | | | | | | |
| | VRQAPG | QAPRLLI | | | | | | |
| | KGLVW | YATSNLV | | | | | | |
| | VSAITSS | AGIPDRF | | | | | | |
| | GGSTYY | SGSGSGT | | | | | | |
| | SDTVKG | DFTLTISR | | | | | | |
| | RFTISRD | LEPEDFA | | | | | | |
| | NAKNTL | VYYCQH | | | | | | |
| | YLQMNS | FSGYHFT | | | | | | |
| | LRAEDT | FGGGTK | | | | | | |
| | AVYYCA | VEIK | | | | | | |
| | RLPDYW | | | | | | | |
| | GQGTLV | | | | | | | |
| | TVSS | | | | | | | |
| SEQ | 1 | 2 | 3 | 4 | 5 | 6 | 7 | 8 |
| ID | | | | | | | | |
| NO | | | | | | | | |
| A9 | QVQLVE | DIQMTQS | GFTFSS | IWYDG | VRSR | QGIR | AAS | LQHN |
| Anti- | SGGGVV | PSSLSAS | YG | SNK | NWNY | NNL | | SYPW |
| Activin | QPGRSL | VGDRVTI | | | DNYY | | | T |
| A | RLSCAA | TCRASQG | | | YGLD | | | |
| | SGFTFSS | IRNNLGW | | | V | | | |
| | YGMHW | YQQKPG | | | | | | |
| | VRQAPG | KAPKRLI | | | | | | |
| | KGLEWV | YAASSLQ | | | | | | |
| | AVIWYD | SGVPSRF | | | | | | |
| | GSNKYH | SGSGSGT | | | | | | |
| | ADSVKG | EFTLTISS | | | | | | |
| | RFTISRD | LQPEDFT | | | | | | |
| | NSKNTL | TYYCLQ | | | | | | |
| | YLQVNS | HNSYPW | | | | | | |
| | LRAEDT | TFGQGTK | | | | | | |
| | AVYYCV | VEIK | | | | | | |
| | RSRNWN | | | | | | | |
| | YDNYYY | | | | | | | |
| | GLDVW | | | | | | | |
| | GQGTTV | | | | | | | |
| | TVSS | | | | | | | |
| SEQ | 11 | 12 | 13 | 14 | 15 | 16 | 17 | 18 |
| ID | | | | | | | | |
| NO | | | | | | | | |

TABLE 2
Anti-myostatin and anti-Activin A Heavy and
Light chain nucleic acids.

| Name | DNA VH | DNA VL |
|---|---|---|
| LIC9 | GAGGTGCAGCTGGTCGAATCCGGC | GAAATCGTTCTTACCCAGTCTC |
| | GGGGGATTAGTCCAACCAGGAGGC | CAGGGACGTTATCTCTGAGCC |
| | TCCCTGAGACTCTCTTGCGCCGCAT | CAGGAGAGAGGGCAACACTAA |
| | CAGGGTTGACCTTCTCACGCTATCC | GCTGTCGAGCGAGTTCCAGTG |
| | TATGTCTTGGGTGAGGCAGGCTCCC | TAAGCTCTAGCTACCTGCATTG |
| | GGGAAAGGTTTGGTGTGGGTGTCAG | GTACCAGCAGAAGCCCGGCCA |
| | CTATTACAAGTAGCGGGGGCAGCA | AGCTCCTCGGTTGCTCATTTAC |
| | CATACTATTCTGACACGGTGAAGGG | GCTACTAGTAACCTGGTGGCC |
| | CCGATTTACAATCAGCAGGGACAAT | GGTATCCCTGACCGCTTCTCAG |
| | GCCAAGAACACCCTGTACCTCCAGA | GGTCAGGCTCAGGAACAGATT |
| | TGAACAGTCTTCGGGCAGAGGATAC | TCACACTCACCATATCCAGACT |
| | TGCCGTTTACTATTGTGCCCGTCTAC | GGAGCCCGAAGACTTTGCCGT |
| | CGGATTACTGGGGACAGGGCACTCT | CTATTATTGCCAGCATTTCTCC |
| | GGTTACCGTATCGTCC | GGGTATCACTTTACTTTCGGAG |
| | | GCGGCACCAAGGTGGAGATTA |
| | | AA |
| SEQ | 9 | 10 |
| ID NO | | |
| A9 | CAGGTCCAGCTCGTCGAATCCGGTG | GACATTCAAATGACCCAGAGT |
| | GAGGCGTCGTGCAGCCAGGACGGT | CCCAGCTCCCTGTCCGCCAGTG |
| | CACTCAGGTTGTCCTGTGCAGCTTC | TCGGTGACAGAGTGACGATAA |
| | AGGATTCACTTTTTCCTCATATGGA | CTTGCCGCGCTAGCCAGGGCA |
| | ATGCACTGGGTAAGACAAGCTCCG | TACGCAATAATCTGGGATGGT |
| | GGGAAAGGCTTGGAGTGGGTGGCT | ACCAGCAAAAACCCGGGAAGG |
| | GTAATCTGGTACGATGGAAGCAATA | CACCTAAGCGGCTCATCTACG |
| | AATATCATGCAGACAGTGTGAAAG | CTGCAAGCTCACTTCAGTCCG |
| | GGAGATTCACTATCTCCAGGGATAA | GGGTCCCAAGTCGATTCAGTG |
| | CTCCAAAAATACCCTGTATCTTCAA | GGAGTGGAAGTGGTACGGAAT |
| | GTGAACAGCCTGCGCGCTGAGGAC | TCACTCTTACAATATCTTCCCT |
| | ACCGCGGTGTACTACTGTGTCCGGT | GCAGCCGGAAGACTTCACTAC |
| | CAAGGAACTGGAATTACGATAACT | GTACTACTGTCTTCAGCACAAT |
| | ATTATTACGGGTTGGACGTTTGGGG | TCTTACCCCTGGACATTTGGCC |
| | ACAAGGCACCACTGTGACTGTGTCA | AGGGCACAAAGGTCGAGATCA |
| | AGT | AA |
| SEQ | 19 | 20 |
| ID NO | | |

TABLE 3
Bispecific Heavy and Light chain amino and nucleic acids,
A9xL1C9kih = Knob in Hole.

| Name | HC 1 | LC 1 | HC 2 | LC 2 |
|---|---|---|---|---|
| A9xL1 | QVQLVESGGGVV | DIQMTQS | EVQLVESGGGLVQP | EIVLTQSPGT |
| C9kih | QPGRSLRLSCAAS | PSSLSASV | GGSLRLSCAASGLT | LSLSPGERAT |
| | GFTFSSYGMHWV | GDRVTIT | FSRYPMSWVRQAPG | LSCRASSSVS |
| | RQAPGKGLEWVA | CRASQGI | KGLVWVSAITSSGG | SSYLHWYQQ |
| | VIWYDGSNKYHA | RNNLGW | STYYSDTVKGRFTIS | KPGQAPRLLI |
| | DSVKGRFTISRDNS | YQQKPGK | RDNAKNTLYLQMN | YATSNLVAGI |
| | KNTLYLQVNSLRA | APKRLIY | SLRAEDTAVYYCAR | PDRFSGSGSG |
| | EDTAVYYCVRSRN | AASSLQS | LPDYWGQGTLVTVS | TDFTLTISRL |
| | WNYDNYYYGLDV | GVPSRFS | SASTKGPSVFPLAPS | EPEDFAVYY |
| | WGQGTTVTVSSAS | GSGSGTE | SKSTSGGTAALGCL | CQHFSGYHF |
| | TKGPSVFPLAPSSK | FTLTISSL | VKDYFPEPVTVSWN | TFGGGTKVEI |
| | STSGGTAALGCLV | QPEDFTT | SGALTSGVHTFPAV | KRTVAAPSV |
| | KDYFPEPVTVSWN | YYCLQHN | LQSSGLYSLSSVVTV | FIFPPSDEQL |
| | SGALTSGVHTFPA | SYPWTFG | PSSSLGTQTYICNVN | KSGTASVVC |
| | VLQSSGLYSLSSV | QGTKVEI | HKPSNTKVDKKVEP | LLNNFYPRE |
| | VTVPSSSLGTQTYI | KRTVAAP | KSCDKTHTCPPCPA | AKVQWKVD |
| | CNVNHKPSNTKVD | SVFIFPPS | PELLGGPSVFLFPPK | NALQSGNSQ |
| | KKVEPKSCDKTHT | DEQLKSG | PKDTLMISRTPEVTC | ESVTEQDSK |
| | CPPCPAPELLGGPS | TASVVCL | VVVDVSHEDPEVKF | DSTYSLSSTL |
| | VFLFPPKPKDTLMI | LNNFYPR | NWYVDGVEVHNAK | TLSKADYEK |
| | SRTPEVTCVVVDV | EAKVQW | TKPREEQYNSTYRV | HKVYACEVT |
| | SHEDPEVKFNWYV | KVDNALQ | VSVLTVLHQDWLN | HQGLSSPVT |
| | DGVEVHNAKTKP | SGNSQES | GKEYKCKVSNKALP | KSFNRGEC |
| | REEQYNSTYRVVS | VTEQDSK | APIEKTISKAKGQPR | |
| | VLTVLHQDWLNG | DSTYSLSS | EPQVYTLPPSRDELT | |
| | KEYKCKVSNKALP | TLTLSKA | KNQVSLTCLVKGFY | |
| | APIEKTISKAKGQP | DYEKHKV | PSDIAVEWESNGQP | |
| | REPQVYTLPPSRDE | YACEVTH | ENNYKTTPPVLDSD | |
| | LTKNQVSLTCLVK | QGLSSPV | GSFFLYSRLTVDKSR | |
| | GFYPSDIAVEWES | TKSFNRG | WQQGNVFSCSVMH | |
| | NGQPENNYKTTPP | EC | EALHNHYTQKSLSL | |
| | VLDSDGSFLLYSK | | SPGK | |
| | LTVDKSRWQQGN | | | |
| | VFSCSVMHEALHN | | | |
| | HYTQKSLSLSPGK | | | |
| SEQ | 21 | 22 | 23 | 24 |
| ID NO | | | | |
| A9xL1 | CAGGTCCAGCTCG | GACATTC | GAGGTGCAGCTGG | gaaatcgttcttaccc |
| C9kih | TCGAATCCGGTGG | AAATGAC | TCGAATCCGGCGG | agtctccagggacgt |
| | AGGCGTCGTGCA | CCAGAGT | GGGATTAGTCCAA | tatctctgagcccag |
| | GCCAGGACGGTC | CCCAGCT | CCAGGAGGCTCCCT | gagagagggcaac |
| | ACTCAGGTTGTCC | CCCTGTC | GAGACTCTCTTGCG | actaagctgtcgagc |
| | TGTGCAGCTTCAG | CGCCAGT | CCGCATCAGGGTTG | gagttccagtgtaag |
| | GATTCACTTTTTC | GTCGGTG | ACCTTCTCACGCTA | ctctagctacctgcat |
| | CTCATATGGAATG | ACAGAGT | TCCTATGTCTTGGG | tggtaccagcagaa |
| | CACTGGGTAAGA | GACGATA | TGAGGCAGGCTCC | gcccggccaagctc |
| | CAAGCTCCGGGG | ACTTGCC | CGGGAAAGGTTTG | ctcggttgctcattta |
| | AAAGGCTTGGAG | GCGCTAG | GTGTGGGTGTCAGC | cgctactagtaacct |
| | TGGGTGGCTGTAA | CCAGGGC | TATTACAAGTAGCG | ggtggccggtatcc |
| | TCTGGTACGATGG | ATACGCA | GGGGCAGCACATA | ctgaccgcttctcag |
| | AAGCAATAAATA | ATAATCT | CTATTCTGACACGG | ggtcaggctcagga |
| | TCATGCAGACAGT | GGGATGG | TGAAGGGCCGATTT | acagatttcacactca |
| | GTGAAAGGGAGA | TACCAGC | ACAATCAGCAGGG | ccatatccagactgg |
| | TTCACTATCTCCA | AAAAACC | ACAATGCCAAGAA | agcccgaagactttg |
| | GGGATAACTCCA | CGGGAAG | CACCCTGTACCTCC | ccgtctattattgcca |
| | AAAATACCCTGTA | GCACCTA | AGATGAACAGTCTT | gcatttctccgggtat |
| | TCTTCAAGTGAAC | AGCGGCT | CGGGCAGAGGATA | cactttactttcggag |
| | AGCCTGCGCGCTG | CATCTAC | CTGCCGTTTACTAT | gcggcaccaaggtg |
| | AGGACACCGCGG | GCTGCAA | TGTGCCCGTCTACC | gagattaaa |
| | TGTACTACTGTGT | GCTCACT | GGATTACTGGGGA | |
| | CCGGTCAAGGAA | TCAGTCC | CAGGGCACTCTGGT | |
| | CTGGAATTACGAT | GGGGTCC | TACCGTATCGTCCG | |
| | AACTATTATTACG | CAAGTCG | CGTCAACAAAAGG | |
| | GGTTGGACGTTTG | ATTCAGT | TCCCTCAGTGTTTC | |
| | GGGACAAGGCAC | GGGAGTG | CTCTGGCCCCATCA | |
| | CACTGTGACTGTG | GAAGTGG | AGCAAGAGCACCT | |
| | TCAAGTGCATCCA | TACGGAA | CCGGCGGCACCGC | |
| | CCAAGGGCCCcagc | TTCACTC | CGCACTGGGATGTC | |
| | gtgttccccctggcccccag | TTACAAT | TCGTGAAGGATTAC | |
| | cagcaagagcaccagcggc | ATCTTCC | TTCCCTGAGCCTGT | |
| | ggcaccgccgccctgggct | CTGCAGC | AACTGTGAGCTGG | |
| | gcctggtgaaggactacttcc | CGGAAGA | AATTCTGGTGCGCT | |
| | ccgagcccgtgaccgtgag | CTTCACT | GACAAGTGGTGTC | |
| | ctggaacagcggcgccctg | ACGTACT | CATACCTTCCCCGC | |
| | accagcggcgtgcacacctt | ACTGTCT | CGTCTTGCAGAGCA | |
| | ccccgccgtgctgcagagca | TCAGCAC | GTGGGCTGTATAGC | |
| | gcggcctgtacagcctgagc | AATTCTT | TTAAGCAGCGTAGT | |
| | agcgtggtgaccgtgcccag | ACCCCTG | GACCGTCCCTAGCT | |
| | cagcagcctgggcacccag | GACATTT | CCTCCCTGGGTACT | |
| | acctacatctgcaacgtgaac | GGCCAGG | CAGACTTATATCTG | |
| | cacaagcccagcaacacca | GCACAAA | CAATGTCAATCATA | |
| | aggtggacaagagagtgga | GGTCGAG | AACCCTCTAATACC | |
| | gcccaagagctgcgacaag | ATCAAAC | AAAGTCGATAAAC | |
| | acccacacctgccccccctg | GAACTGT | GCGTGGAGCCTAA | |
| | ccccgcccccgagctgctgg | GGCTGCA | ATCTTGTGATAAAA | |
| | gcggccccagcgtgttcctgt | CCATCTG | CCCATACCTGCCCA | |
| | tcccccccaagcccaaggac | TCTTCAT | CCATGTCCCGCCCC | |
| | accctgatgatcagcagaac | CTTCCCG | TGAGCTGCTCGGCG | |
| | ccccgaggtgacctgcgtgg | CCATCTG | GTCCATCCGTCTTC | |
| | tggtggacgtgagccacgag | ATGAGCA | CTGTTTCCTCCAAA | |
| | gaccccgaggtgaagttcaa | GTTGAAA | ACCAAAGGACACA | |
| | ctggtacgtggacggcgtgg | TCTGGAA | TTGATGATTAGTAG | |
| | aggtgcacaacgccaagac | CTGCCTC | GACACCCGAGGTC | |
| | caagcccagagaggagcag | TGTTGTG | ACCTGTGTGGTGGT | |
| | tacgcgagcacctacagagt | TGCCTGC | TGACGTTTCCCACG | |
| | ggtgagcgtgctgaccgtgc | TGAATAA | AGGACCCAGAGGT | |
| | tgcaccaggactggctgaac | CTTCTAT | CAAATTCAACTGGT | |
| | ggcaaggagtacaagtgca | CCCAGAG | ATGTGGACGGCGT | |
| | aggtgagcaacaaggccct | AGGCCAA | GGAGGTCCACAAC | |
| | gcccgcccccatcgagaag | AGTACAG | GCTAAAACCAAAC | |
| | accatcagcaaggccaagg | TGGAAGG | CCAGAGAAGAACA | |
| | gccagcccagagagcccca | TGGATAA | GTACGCCTCTACTT | |
| | ggtgtacaccctgcccccca | CGCCCTC | ATAGAGTTGTGAG | |
| | gcagagaggagatgaccaa | CAATCGG | CGTCCTCACGGTGC | |
| | gaaccaggtgagcctgacct | GTAACTC | TGCACCAGGATTG | |
| | gcctggtgaagggcttctacc | CCAGGAG | GCTGAATGGCAAG | |
| | ccagcgacatcgccgtgga | AGTGTCA | GAGTATAAGTGCA | |
| | gtgggagagcaacggccag | CAGAGCA | AAGTCTCTAATAAA | |
| | cccgagaacaactacaagac | GGACAGC | GCACTCCCTGCGCC | |
| | caccccccccgtgctggaca | AAGGACA | AATAGAGAAGACA | |
| | gcgacggcagcttcctgctgt | GCACCTA | ATTAGCAAGGCAA | |
| | acagcaagctgaccgtggac | CAGCCTC | AGGGACAACCACG | |
| | aagagcagatggcagcagg | AGCAGCA | CGAACCTCAGGTGT | |
| | gcaacgtgttcagctgcagc | CCCTGAC | ACACTCTCCCCCCT | |
| | gtgatgcacgaggccctgca | GCTGAGC | AGCCGGGAAGAGA | |
| | caaccactacacccagaaga | AAAGCAG | TGACAAAAAATCA | |
| | gcctgagcctgagccccgg | ACTACGA | GGTTTCTTTAACAT | |
| | caag | GAAACAC | GTCTGGTGAAAGG | |
| | | AAAGTCT | ATTTTACCCTTCCG | |
| | | ACGCCTG | ACATCGCGGTCGA | |
| | | CGAAGTC | ATGGGAGTCAAAT | |
| | | ACCCATC | GGACAACCCGAGA | |
| | | AGGGCCT | ATAATTACAAGAC | |
| | | GAGCTCG | AACACCACCAGTG | |
| | | CCCGTCA | TTAGACTCCGATGG | |
| | | CAAAGAG | CTCCTTCTTTCTGT | |
| | | CTTCAAC | ACAGCAGGCTGAC | |
| | | AGGGGA | GGTGGACAAGAGC | |
| | | GAGTGT | CGCTGGCAACAGG | |
| | | | GGAATGTCTTCTCT | |
| | | | TGCTCTGTGATGCA | |
| | | | TGAGGCACTCCAC | |
| | | | AATCATTATACGCA | |
| | | | GAAAAGTCTCTCTC | |
| | | | TCTCTCCAGGCAAA | |
| | 25 | 26 | 27 | 28 |

TABLE 4
Bispecific Heavy and Light chain amino and nucleic
acids, A9xL1C9FiS = IgG-ScFv.

| | HC 1 | LC 1 |
|---|---|---|
| A9xL1C | QVQLVESGGGVVQPGRSLRLSCAASGFTF | DIQMTQSPSSLSASVGD |
| 9FiS | SSYGMHWVRQAPGKGLEWVAVIWYDGS | RVTITCRASQGIRNNLG |
| | NKYHADSVKGRFTISRDNSKNTLYLQVNS | WYQQKPGKAPKRLIYA |
| | LRAEDTAVYYCVRSRNWNYDNYYYGLD | ASSLQSGVPSRFSGSGSG |
| | VWGQGTTVTVSSASTKGPSVFPLAPCSRS | TEFTLTISSLQPEDFTTY |
| | TSESTAALGCLVKDYFPEPVTVSWNSGAL | YCLQHNSYPWTFGQGT |
| | TSGVHTFPAVLQSSGLYSLSSVVTVPSSSL | KVEIKRTVAAPSVFIFPP |
| | GTKTYTCNVDHKPSNTKVDKRVESKYGP | SDEQLKSGTASVVCLLN |
| | PCPPCPAPEFLGGPSVFLFPPKPKDTLMISR | NFYPREAKVQWKVDNA |
| | TPEVTCVVVDVSQEDPEVQFNWYVDGVE | LQSGNSQESVTEQDSKD |
| | VHNAKTKPREEQFNSTYRVVSVLTVLHQ | STYSLSSTLTLSKADYEK |
| | DWLNGKEYKCKVSNKGLPSSIEKTISKAK | HKVYACEVTHQGLSSPV |
| | GQPREPQVYTLPPSQEEMTKNQVSLTCLV | TKSFNRGEC |
| | KGFYPSDIAVEWESNGQPENNYKTTPPVL | |
| | DSDGSFFLYSRLTVDKSRWQEGNVFSCSV | |
| | LHEALHSHYTQKSLSLSLGGGGGSGGGGS | |
| | GGGGSEVQLVESGGGLVQPGGSLRLSCAA | |
| | SGLTFSRYPMSWVRQAPGKGLVWVSAITS | |
| | SGGSTYYSDTVKGRFTISRDNAKNTLYLQ | |
| | MNSLRAEDTAVYYCARLPDYWGQGTLVT | |
| | VSSGGGGSGGGGSGGGGSEIVLTQSPGTL | |
| | SLSPGERATLSCRASSSVSSSYLHWYQQKP | |
| | GQAPRLLIYATSNLVAGIPDRFSGSGSGTD | |
| | FTLTISRLEPEDFAVYYCQHFSGYHFTFGG | |
| | GTKVEIK | |
| SEQ ID | 29 | 30 |
| NO | | |
| A9xL1C | CAGGTACAATTGGTAGAGAGCGGAGGA | GACATTCAAATGACCC |
| 9FiS | GGCGTTGTGCAGCCAGGTCGGTCTCTCC | AGAGTCCCAGCTCCCT |
| | GACTGTCCTGTGCCGCCTCTGGCTTCAC | GTCCGCCAGTGTCGGT |
| | GTTCTCCTCATATGGAATGCATTGGGTC | GACAGAGTGACGATAA |
| | CGTCAAGCTCCAGGGAAAGGACTCGAG | CTTGCCGCGCTAGCCA |
| | TGGGTCGCCGTCATTTGGTACGATGGTA | GGGCATACGCAATAAT |
| | GCAATAAGTATCATGCCGATAGCGTCAA | CTGGGATGGTACCAGC |
| | GGGCAGATTTACCATCAGTAGAGACAAC | AAAAACCCGGGAAGGC |
| | AGTAAGAACACTTTGTATCTGCAAGTGA | ACCTAAGCGGCTCATC |
| | ATAGTCTGCGGGCAGAGGACACGGCAG | TACGCTGCAAGCTCAC |
| | TGTACTACTGTGTAAGGTCTAGGAATTG | TTCAGTCCGGGGTCCC |
| | GAATTATGACAACTACTATTATGGCCTC | AAGTCGATTCAGTGGG |
| | GATGTTTGGGGCCAGGGGACAACTGTCA | AGTGGAAGTGGTACGG |
| | CCGTCTCATCCGCTAGTACAAAAGGACC | AATTCACTCTTACAATA |
| | TAGCGTGTTCCCTTTGGCTCCCTGTAGTC | TCTTCCCTGCAGCCGGA |
| | GTTCAACCTCTGAAAGCACAGCAGCGCT | AGACTTCACTACGTACT |
| | GGGGTGTTTGGTCAAGGACTATTTTCCC | ACTGTCTTCAGCACAAT |
| | GAACCCGTGACCGTCAGCTGGAATTCTG | TCTTACCCCTGGACATT |
| | GGGCGTTGACCTCTGGAGTTCATACATT | TGGCCAGGGCACAAAG |
| | TCCCGCTGTGCTCCAAAGTTCCGGCTTG | GTCGAGATCAAACGAA |
| | TATTCCCTTAGTAGCGTTGTTACCGTTCC | CTGTGGCTGCACCATCT |
| | ATCATCCAGCTTGGGCACCAAAACTTAT | GTCTTCATCTTCCCGCC |
| | ACCTGTAATGTTGATCATAAACCCTCAA | ATCTGATGAGCAGTTG |
| | ATACAAAGGTCGATAAGAGGGTCGAAA | AAATCTGGAACTGCCT |
| | GCAAATACGGGCCCCCTTGTCCGCCCTG | CTGTTGTGTGCCTGCTG |
| | TCCAGCGCCCGAGTTTCTGGGCGGGCCT | AATAACTTCTATCCCAG |
| | TCTGTGTTCTTGTTCCCGCCAAAACCCA | AGAGGCCAAAGTACAG |
| | AAGATACTCTCATGATTAGCCGAACTCC | TGGAAGGTGGATAACG |
| | CGAGGTGACATGCGTCGTCGTTGATGTC | CCCTCCAATCGGGTAA |
| | TCTCAAGAAGATCCTGAAGTGCAATTTA | CTCCCAGGAGAGTGTC |
| | ATTGGTATGTTGATGGCGTGGAAGTACA | ACAGAGCAGGACAGCA |
| | CAATGCTAAGACCAAGCCCCGCGAGGA | AGGACAGCACCTACAG |
| | ACAATTTAATAGCACTTACCGTGTGGTG | CCTCAGCAGCACCCTG |
| | AGCGTTCTCACTGTCCTGCATCAAGACT | ACGCTGAGCAAAGCAG |
| | GGTTGAATGGTAAAGAATATAAATGCA | ACTACGAGAAACACAA |
| | AAGTGTCCAATAAAGGACTGCCCAGCA | AGTCTACGCCTGCGAA |
| | GCATCGAAAAGACCATATCCAAAGCTA | GTCACCCATCAGGGCC |
| | AGGGGCAACCGCGAGAACCTCAAGTTT | TGAGCTCGCCCGTCAC |
| | ATACGCTCCCACCATCCCAAGAAGAAAT | AAAGAGCTTCAACAGG |
| | GACAAAGAACCAAGTGTCCCTTACTTGC | GGAGAGTGT |
| | TTGGTGAAGGGCTTTTACCCTTCAGATA | |
| | TAGCAGTAGAATGGGAAAGCAATGGCC | |
| | AACCTGAAAACAATTATAAAACAACTCC | |
| | CCCAGTACTGGATAGTGATGGCTCTTTC | |
| | TTCCTTTATAGCCGTCTTACAGTTGACAA | |
| | GAGCCGCTGGCAAGAAGGCAATGTGTTC | |
| | TCTTGTTCAGTCCTGCACGAAGCGCTCC | |
| | ACTCTCATTATACTCAGAAGAGTTTGTC | |
| | ACTCAGTTTGGGGGGTGGTGGCGGTTCT | |
| | GGTGGTGGTGGCTCTGGTGGCGGCGGTT | |
| | CTGAAGTCCAGCTGGTTGAATCCGGCGG | |
| | TGGTCTCGTCCAGCCGGGTGGATCTCTG | |
| | AGGTTGAGCTGTGCTGCAAGCGGTCTGA | |
| | CCTTTTCTCGATACCCGATGTCCTGGGTC | |
| | CGCCAAGCTCCAGGGAAAGGTCTGGTGT | |
| | GGGTTTCAGCCATTACGAGCTCCGGCGG | |
| | CTCAACCTACTATAGTGATACCGTGAAG | |
| | GGTAGATTCACGATCTCCAGAGACAATG | |
| | CTAAGAATACCCTGTATCTTCAAATGAA | |
| | TTCTCTGAGGGCTGAGGATACAGCTGTT | |
| | TATTATTGTGCAAGGCTCCCTGACTATT | |
| | GGGGACAAGGCACACTCGTCACAGTCTC | |
| | AAGTGGCGGTGGTGGTTCTGGCGGCGGT | |
| | GGAAGCGGCGGTGGCGGAAGCGAAATC | |
| | GTGTTGACCCAGTCCCCAGGCACTCTGT | |
| | CTCTGTCTCCAGGTGAGCGAGCAACCCT | |
| | GTCTTGCAGGGCTTCATCATCCGTGAGT | |
| | TCTTCCTACCTGCACTGGTACCAACAAA | |
| | AGCCTGGACAGGCCCCTCGTCTCCTCAT | |
| | CTACGCTACATCCAATCTCGTTGCTGGT | |
| | ATTCCAGACCGATTTTCTGGCTCCGGGA | |
| | GCGGCACAGATTTTACTTTGACAATCTC | |
| | TCGGCTGGAACCCGAGGACTTCGCCGTG | |
| | TATTATTGTCAACACTTCAGCGGATATC | |
| | ATTTTACCTTTGGTGGTGGAACAAAAGT | |
| | GGAAATCAAATAA | |
| SEQ ID | 31 | 32 |
| NO | | |

### EMBODIMENTS

Embodiment 1. A bispecific anti-myostatin and anti-Activin A antibody or antigen binding fragments thereof, comprising: a first antibody or antigen binding fragments thereof that binds to myostatin; a second antibody or antigen binding fragments thereof that binds to Activin A; wherein the first and second antibody or antigen binding fragments are selected to modulate or bind to both myostatin and anti-Activin A.

Embodiment 2. The bispecific antibody or antigen binding fragments of embodiment 1, wherein the anti-myostatin antibody or binding fragment thereof comprising: a heavy chain variable domain (VH) complementarity determining region 1 (CDR1) comprising the amino acid sequence of any one of the following SEQ ID NO: 3; a VH CDR2 of SEQ ID NO: 4; and a VH CDR3 of SEQ ID NO: 5; and a light chain variable domain (VL) CDR1 of SEQ ID NO: 6; a VL CDR2 of SEQ ID NO: 7; and a VL CDR3 of SEQ ID NO: 8.

Embodiment 3. The bispecific antibody or antigen binding fragments of embodiments 1 or 2, wherein the anti-Activin A antibody or binding fragment thereof comprising: a heavy chain variable domain (VH) complementarity determining region 1 (HCDR1) comprising the amino acid sequence of SEQ ID NO: 13; a HCDR2 of SEQ ID NO: 14; and a HCDR3 of SEQ ID NO: 15; and a light chain variable domain (VL) complementarity determining region 1 (LCDR1) comprising the amino acid sequence of SEQ ID NO: 16; LCDR2 of SEQ ID NO: 17; and LCDR3 of SEQ ID NO: 18.

Embodiment 4. The bispecific antibody or antigen binding fragments of any one of embodiments 1 to 3, wherein the anti-myostatin antibody or binding fragment thereof comprises:
a heavy chain having at least 80%, or at least 85%, or at least 90%, 91%, 92%, 93%, 94%, 95%, 96%, 97%, 98%, 99%, or 100% sequence identity to SEQ ID NO: 1, and
a light chain having at least 80%, or at least 85%, or at least 90%, 91%, 92%, 93%, 94%, 95%, 96%, 97%, 98%, 99%, or 100% sequence identity to SEQ ID NO: 2.

Embodiment 5. The bispecific antibody or antigen binding fragments of any one of embodiments 1 to 4, wherein the anti-Activin A antibody or binding fragment thereof comprises:
a heavy chain having at least 80%, or at least 85%, or at least 90%, 91%, 92%, 93%, 94%, 95%, 96%, 97%, 98%, 99%, or 100% sequence identity to SEQ ID NO: 19, and
a light chain having at least 80%, or at least 85%, or at least 90%, 91%, 92%, 93%, 94%, 95%, 96%, 97%, 98%, 99%, or 100% sequence identity to SEQ ID NO: 20.

Embodiment 6. The bispecific antibody or antigen binding fragments of any one of embodiments 1 to 5, wherein antibody or binding fragment thereof is encoded by a nucleic acid that has at least 80%, or at least 85%, or at least 90%, 91%, 92%, 93%, 94%, 95%, 96%, 97%, 98%, 99%, or 100% sequence identity to SEQ ID NOS: 21, 22, 23, and 24.

Embodiment 7. The bispecific antibody or antigen binding fragments of any one of embodiments 1 to 6, wherein the bispecific antibody or binding fragment thereof is encoded by a nucleic acid that has at least 80%, or at least 85%, or at least 90%, 91%, 92%, 93%, 94%, 95%, 96%, 97%, 98%, 99% or 100% to SEQ ID NOS: 29 and 30.

Embodiment 8. The bispecific antibody or antigen binding fragments of any one of embodiments 1 to 7, wherein the bispecific antibody or binding fragment thereof is encoded by a nucleic acid that has at least 80%, or at least 85%, or at least 90%, 91%, 92%, 93%, 94%, 95%, 96%, 97%, 98%, 99% or 100% to SEQ ID NOS: 9 or 19, and a VL nucleic acid that has at least 80%, or at least 85%, or at least 90%, 91%, 92%, 93%, 94%, 95%, 96%, 97%, 98%, 99% or 100% to SEQ ID NOS: 10 or 20.

Embodiment 9. The bispecific antibody or antigen binding fragments of any one of embodiments 1 to 8, wherein the bispecific antibody or binding fragment thereof is encoded by a nucleic acid that has at least 80%, or at least 85%, or at least 90%, 91%, 92%, 93%, 94%, 95%, 96%, 97%, 98%, 99% or 100% to SEQ ID NOS: 26 and 26; 27 and 28; or 31 and 32.

Embodiment 10. The bispecific antibody or antigen binding fragments of any one of embodiments 1 to 9, wherein the Fc domain of any one of the following: human IgG1, human IgG2, human IgG3, human IgG4, or mutants thereof.

Embodiment 11. The bispecific antibody or antigen binding fragments of any one of embodiments 1 to 10, wherein the bispecific antibody comprises a bispecific knob-in-hole, IgG-ScFv, ScFv-IgG, scFV-Fc-ScFv, or scFv-scFv-Fc.

Embodiment 12. The bispecific antibody or antigen binding fragments of any one of embodiments 1 to 11, further comprising a linker between the first and second antibody or antigen binding fragments thereof.

Embodiment 13. The bispecific antibody or antigen binding fragments of any one of embodiments 1 to 12, further comprising a payload.

Embodiment 14. The bispecific antibody or antigen binding fragments of any one of embodiments 1 to 13, further comprising an anti-cancer drug.

Embodiment 15. A polynucleotide encoding the anti-myostatin and anti-Activin A antibody or antigen binding fragments of embodiment 1.

Embodiment 16. An expression vector comprising a nucleic acid encoding bispecific anti-myostatin and anti-Activin A antibody or antigen binding fragments of embodiment 15.

Embodiment 17. A host cell comprising the vector of embodiment 16.

Embodiment 18. A method of making a bispecific anti-myostatin and anti-Activin A antibody or antigen binding fragments comprising: culturing a cell comprising the polynucleotide of claim 15, in a culture medium, expressing the polynucleotide, and collecting the bispecific anti-myostatin and anti-Activin A antibody or antigen binding fragments thereof from the cells or culture medium.

Embodiment 19. A method of treating a disease or condition of obesity, muscle-related, bone growth, cachexia, or cancer in a subject, comprising: administering the bispecific anti-myostatin and anti-Activin A antibody or antigen binding fragments of any one of embodiments 1 to 14, the polynucleotide of embodiment 15, or the expression vector of embodiment 16 to the patient.

Embodiment 20. The method of embodiment 19, wherein the disease or condition is at least one of: obesity, adipose tissue disorder, metabolic syndrome, hyperphagia-associated obesity, dyslipidemia, diabetes, visceral adiposity, sarcopenic obesity, or cachexia in obese patients.

Embodiment 21. The method of embodiment 19, wherein the disease or condition is selected from loss of body weight, loss of muscle mass, or loss of fat mass.

Embodiment 22. The method of embodiment 19, wherein the cancer is selected from: gonadal tumor, ovarian cancer, benign prostatic hyperplasia, prostate intraepithelial neoplasia, or prostate cancer, or wherein the tumor is bladder cancer, Wilm's tumor, pancreatic cancer, breast cancer, bone cancer, lung cancer, colorectal cancer, cervical cancer, synovial sarcoma, vasoactive intestinal peptide secreting tumors, glioblastoma, medulloblastoma, head and neck squamous cell cancer, oral cancer, oral leukoplakia, anal cancer, esophageal cancer, gastric cancer, bone cancer, or metastatic cancer.

Embodiment 23. The method of embodiment 19, wherein the disease or condition of bone growth is selected from at least one of: metabolic disorders selected from type 2 diabetes, impaired glucose tolerance, syndrome X, insulin resistance induced by trauma, burns or nitrogen imbalance; adipose tissue disorders (obesity); muscle and neuromuscular disorders such as muscular dystrophy (Duchenne muscular dystrophy); amyotrophic lateral sclerosis (ALS); muscle atrophy; organ atrophy; frailty; carpal tunnel syndrome; congestive obstructive pulmonary disease; and sarcopenia, cachexia and other muscle wasting syndromes; osteoporosis, osteoporosis in elderly and/or postmenopausal women; glucocorticoid-induced osteoporosis; osteopenia; osteoarthritis; and osteoporosis-related fractures; low bone mass due to chronic glucocorticoid therapy, premature gonadal failure, androgen suppression, vitamin D deficiency, secondary hyperparathyroidism, nutritional deficiencies, and anorexia nervosa.

Embodiment 24. The method of embodiment 19, wherein the muscle-related diseases or conditions are selected from at least one of: neuromuscular disorders, muscular dystrophy and muscle atrophy, congestive obstructive pulmonary disease, muscle wasting associated with diabetes, bone degenerative disease, osteoporosis; musculodegenerative and neuromuscular disorders, tissue repair, wound healing, neurodegenerative diseases, amyotrophic lateral sclerosis, immunologic disorders, disorders related to abnormal proliferation or function of lymphocytes, and obesity or disorders related to abnormal proliferation of adipocytes.

Embodiment 25. The method of any one of embodiments 19 to 24, wherein the subject is human.

Embodiment 26. The method of any one of embodiments 19 to 25, wherein the bispecific antibody or antigen binding fragment having a predetermined activity is used during a first phase of treatment, and a second bispecific antibody or antigen binding fragment having a predetermined activity is used during a second phase of treatment, and optionally using one or more different bispecific antibody or antigen binding fragment having a predetermined activity is used during a first phase of treatment for any additional phases of treatment.

Embodiment 27. The method of any one of embodiments 19 to 26, further comprising a payload.

Embodiment 28. The method of any one of embodiments 19 to 27, further comprising an anti-cancer drug.

Embodiment 29. A method of selecting a bispecific anti-myostatin and anti-Activin A antibody or antigen binding fragments comprising: selecting an antibody against the antigen; generating the bispecific anti-myostatin and anti-Activin A antibody or antigen binding fragments; contacting the bispecific anti-myostatin and anti-Activin A antibody or antigen binding fragments under conditions in which a muscle cell expresses myostatin and Activin A; measuring the effect of the bispecific anti-myostatin and anti-Activin A antibody or antigen binding fragments on the muscle cell; and selecting the combination of bispecific anti-myostatin and anti-Activin A antibody or antigen binding fragments having a predetermined activity level.

Embodiment 30. The method of embodiment 27, wherein the bispecific antibody comprises bispecific knob-in-hole, IgG-ScFv, ScFv-IgG, scFV-Fc-ScFv, or scFv-scFv-Fc.

Embodiment 31. The method of embodiment 27, wherein the activity is selected from at least one of: cachexia, loss of body weight, loss of muscle mass, or loss of fat mass; gonadal tumor, ovarian cancer, benign prostatic hyperplasia, prostate intraepithelial neoplasia, or prostate cancer, or wherein the tumor is bladder cancer, Wilm's tumor, pancreatic cancer, breast cancer, bone cancer, lung cancer, colorectal cancer, cervical cancer, synovial sarcoma, vasoactive intestinal peptide secreting tumors, glioblastoma, medulloblastoma, head and neck squamous cell cancer, oral cancer, oral leukoplakia, anal cancer, esophageal cancer, gastric cancer, bone cancer, or metastatic cancer; wherein the disease or condition of bone growth is selected from at least one of: metabolic disorders selected from type 2 diabetes, impaired glucose tolerance, syndrome X, insulin resistance induced by trauma, burns or nitrogen imbalance; adipose tissue disorders (obesity); muscle and neuromuscular disorders such as muscular dystrophy (Duchenne muscular dystrophy); amyotrophic lateral sclerosis (ALS); muscle atrophy; organ atrophy; frailty; carpal tunnel syndrome; congestive obstructive pulmonary disease; and sarcopenia, cachexia and other muscle wasting syndromes; osteoporosis, osteoporosis in elderly and/or postmenopausal women; glucocorticoid-induced osteoporosis; osteopenia; osteoarthritis; and osteoporosis-related fractures; low bone mass due to chronic glucocorticoid therapy, premature gonadal failure, androgen suppression, vitamin D deficiency, secondary hyperparathyroidism, nutritional deficiencies, and anorexia nervosa; or wherein the muscle-related diseases or conditions are selected from at least one of: neuromuscular disorders, muscular dystrophy and muscle atrophy, congestive obstructive pulmonary disease, muscle wasting associated with COPD, muscle wasting syndrome, sarcopenia, cachexia, adipose tissue disorders, type 2 diabetes, bone degenerative disease, osteoporosis; musculodegenerative and neuromuscular disorders, tissue repair, wound healing, neurodegenerative diseases, amyotrophic lateral sclerosis, immunologic disorders, disorders related to abnormal proliferation or function of lymphocytes, and obesity or disorders related to abnormal proliferation of adipocytes.

Embodiment 32. The method of any one of embodiments 27 to 31, wherein the bispecific anti-myostatin and anti-Activin A antibody or antigen binding fragments thereof are selected from: a heavy chain variable domain (VH) complementarity determining region 1 (CDR1), CDR2, and CDR3 comprising the amino acid sequence of any one of the following SEQ ID NOs: 3, 4, 5; and 13, 14, 15; and a light chain variable domain (VL) CDR1, CDR2, and CDR3 comprising the amino acid sequence of any one of the following SEQ ID NOs: 6, 7 and 8; and 16, 17, and 18.

Embodiment 33. The method of any one of embodiments 27 to 32, wherein the bispecific anti-myostatin and anti-Activin A antibody or antigen binding fragments thereof comprises: a heavy chain comprises at least 80%, or at least 85%, or at least 90%, 91%, 92%, 93%, 94%, 95%, 96%, 97%, 98%, 99% or 100% to SEQ ID NOs: 1 and 2, and a light chain comprises at least 80%, or at least 85%, or at least 90%, 91%, 92%, 93%, 94%, 95%, 96%, 97%, 98%, 99% or 100% to SEQ ID NOs: 11 and 12.

Embodiment 34. The method of any one of embodiments 27 to 33, wherein the bispecific anti-myostatin and anti-Activin A antibody or antigen binding fragments antibody or binding fragment thereof is encoded by a nucleic acid that comprises: a VH comprising a nucleic acid sequence that comprises at least 80%, or at least 85%, or at least 90%, 91%, 92%, 93%, 94%, 95%, 96%, 97%, 98%, 99% or 100% to SEQ ID NOS: 9 and 19, and a VL comprising a nucleic acid sequence that comprises at least 80%, or at least 85%, or at least 90%, 91%, 92%, 93%, 94%, 95%, 96%, 97%, 98%, 99% or 100% to SEQ ID NOs: 10 and 20.

Embodiment 35. The method of any one of embodiments 27 to 34, wherein the bispecific anti-myostatin and anti-Activin A antibody or antigen binding fragments comprises: a heavy chain variable domain (VH) comprising SEQ ID NOS: 21, 23, or 29; and a light chain variable domain (VL) comprising SEQ ID NO: 22, 24, and 30.

It is contemplated that any aspects of the disclosure discussed in this specification can be implemented with respect to any method, kit, reagent, or composition of the disclosure, and vice versa. Furthermore, compositions of the disclosure can be used to achieve methods of the disclosure.

It will be understood that particular aspects described herein are shown by way of illustration and not as limitations of the disclosure. The principal features of this disclosure can be employed in various aspects without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In aspects of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the disclosure(s) set out in any claims that may issue from this disclosure. Specifically, and by way of example, although the headings refer to a “Field of Invention,” such claims should not be limited by the language under this heading to describe the so-called technical field. Further, a description of technology in the “Background” section is not to be construed as an admission that technology is prior art to any disclosure(s) in this disclosure. Neither is the “Summary” to be considered a characterization of the disclosure(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure but should not be constrained by the headings set forth herein.

All of the compositions and/or methods disclosed and claimed herein can be made and executed without undue experimentation in light of the present disclosure. While the compositions and methods of this disclosure have been described in terms of preferred aspects, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A bispecific anti-myostatin and anti-Activin A antibody or antigen binding fragments thereof, comprising:
a first antibody or antigen binding fragments thereof that binds to myostatin;
a second antibody or antigen binding fragments thereof that binds to Activin A;
wherein the first and second antibody or antigen binding fragments are selected to modulate or bind to both myostatin and anti-Activin A.

2. The bispecific antibody or antigen binding fragments of claim 1, wherein the anti-myostatin antibody or binding fragment thereof comprising:
a heavy chain variable domain (VH) complementarity determining region 1 (CDR1) comprising the amino acid sequence of any one of the following SEQ ID NO: 3; a VH CDR2 of SEQ ID NO: 4; and a VH CDR3 of SEQ ID NO: 5; and
a light chain variable domain (VL) CDR1 of SEQ ID NO: 6; a VL CDR2 of SEQ ID NO: 7; and a VL CDR3 of SEQ ID NO: 8.

3. The bispecific antibody or antigen binding fragments of claim 1, wherein the anti-Activin A antibody or binding fragment thereof comprising:
a heavy chain variable domain (VH) complementarity determining region 1 (HCDR1) comprising the amino acid sequence of SEQ ID NO: 13; a HCDR2 of SEQ ID NO: 14; and a HCDR3 of SEQ ID NO: 15; and
a light chain variable domain (VL) complementarity determining region 1 (LCDR1) comprising the amino acid sequence of SEQ ID NO: 16; LCDR2 of SEQ ID NO: 17; and
LCDR3 of SEQ ID NO: 18.

4. The bispecific antibody or antigen binding fragments of claim 1, wherein the anti-myostatin antibody or binding fragment thereof comprises:
a heavy chain having at least 80%, or at least 85%, or at least 90%, 91%, 92%, 93%, 94%, 95%, 96%, 97%, 98%, 99%, or 100% sequence identity to SEQ ID NO: 1, and
a light chain having at least 80%, or at least 85%, or at least 90%, 91%, 92%, 93%, 94%, 95%, 96%, 97%, 98%, 99%, or 100% sequence identity to SEQ ID NO: 2.

5. The bispecific antibody or antigen binding fragments of claim 1, wherein the anti-Activin A antibody or binding fragment thereof comprises:
a heavy chain having at least 80%, or at least 85%, or at least 90%, 91%, 92%, 93%, 94%, 95%, 96%, 97%, 98%, 99%, or 100% sequence identity to SEQ ID NO: 19, and
a light chain having at least 80%, or at least 85%, or at least 90%, 91%, 92%, 93%, 94%, 95%, 96%, 97%, 98%, 99%, or 100% sequence identity to SEQ ID NO: 20.

6. The bispecific antibody or antigen binding fragments of claim 1, wherein at least one of:
the antibody or binding fragment thereof is encoded by a nucleic acid that has at least 80%, or at least 85%, or at least 90%, 91%, 92%, 93%, 94%, 95%, 96%, 97%, 98%, 99%, or 100% sequence identity to SEQ ID NOS: 21, 22, 23, and 24; or
the bispecific antibody or binding fragment thereof is encoded by a nucleic acid that has at least 80%, or at least 85%, or at least 90%, 91%, 92%, 93%, 94%, 95%, 96%, 97%, 98%, 99% or 100% to SEQ ID NOS: 29 and 30.

7. The bispecific antibody or antigen binding fragments of claim 1, wherein at least one of:
the bispecific antibody or binding fragment thereof is encoded by a nucleic acid that has at least 80%, or at least 85%, or at least 90%, 91%, 92%, 93%, 94%, 95%, 96%, 97%, 98%, 99% or 100% to SEQ ID NOS: 9 or 19, and a VL nucleic acid that has at least 80%, or at least 85%, or at least 90%, 91%, 92%, 93%, 94%, 95%, 96%, 97%, 98%, 99% or 100% to SEQ ID NOS: 10 or 20; or
wherein the bispecific antibody or binding fragment thereof is encoded by a nucleic acid that has at least 80%, or at least 85%, or at least 90%, 91%, 92%, 93%, 94%, 95%, 96%, 97%, 98%, 99% or 100% to SEQ ID NOS: 26 and 26; 27 and 28; or 31 and 32.

8. The bispecific antibody or antigen binding fragments of claim 1, wherein at least one of:
the Fc domain of any one of the following: human IgG1, human IgG2, human IgG3, human IgG4, or mutants thereof;
the bispecific antibody comprises a bispecific knob-in-hole, IgG-ScFv, ScFv-IgG, scFV-Fc-ScFv, or scFv-scFv-Fc;
further comprising a linker between the first and second antibody or antigen binding fragments thereof;
further comprising a payload; or
further comprising an anti-cancer drug.

9. A polynucleotide encoding the anti-myostatin and anti-Activin A antibody or antigen binding fragments comprising:
a first antibody or antigen binding fragments thereof that binds to myostatin;
a second antibody or antigen binding fragments thereof that binds to Activin A;
wherein the first and second antibody or antigen binding fragments are selected to modulate or bind to both myostatin and anti-Activin A.

10. The polynucleotide of claim 9, further comprising an expression vector comprising a nucleic acid encoding bispecific anti-myostatin and anti-Activin A antibody or antigen binding fragments.

11. The polynucleotide of claim 9, further comprising a host cell comprising an expression vector comprising the nucleic acid encoding bispecific anti-myostatin and anti-Activin A antibody or antigen binding fragments.

12. A method of making a bispecific anti-myostatin and anti-Activin A antibody or antigen binding fragments comprising:
culturing a cell comprising a polynucleotide expressing polynucleotide encoding the anti-myostatin and anti-Activin A antibody or antigen binding fragments encoding:
a first antibody or antigen binding fragments thereof that binds to myostatin;
a second antibody or antigen binding fragments thereof that binds to Activin A;
wherein the first and second antibody or antigen binding fragments are selected to modulate or bind to both myostatin and anti-Activin A, in a culture medium, expressing the polynucleotide, and collecting the bispecific anti-myostatin and anti-Activin A antibody or antigen binding fragments thereof from the cells or culture medium.

13. A method of treating a disease or condition of obesity, muscle-related, bone growth, cachexia, or cancer in a mammal or human subject, comprising: administering a bispecific anti-myostatin and anti-Activin A antibody or antigen binding fragments comprising:
a first antibody or antigen binding fragments thereof that binds to myostatin;
a second antibody or antigen binding fragments thereof that binds to Activin A;
wherein the first and second antibody or antigen binding fragments are selected to modulate or bind to both myostatin and anti-Activin A, and optionally comprises a payload or an anti-cancer drug.

14. The method of claim 13, wherein the disease or condition is at least one of: obesity, adipose tissue disorder, metabolic syndrome, hyperphagia-associated obesity, dyslipidemia, diabetes, visceral adiposity, sarcopenic obesity, or cachexia in obese patients.

15. The method of claim 13, wherein the disease or condition is selected from loss of body weight, loss of muscle mass, or loss of fat mass.

16. The method of claim 13, wherein the cancer is selected from: gonadal tumor, ovarian cancer, benign prostatic hyperplasia, prostate intraepithelial neoplasia, or prostate cancer, or wherein the tumor is bladder cancer, Wilm's tumor, pancreatic cancer, breast cancer, bone cancer, lung cancer, colorectal cancer, cervical cancer, synovial sarcoma, vasoactive intestinal peptide secreting tumors, glioblastoma, medulloblastoma, head and neck squamous cell cancer, oral cancer, oral leukoplakia, anal cancer, esophageal cancer, gastric cancer, bone cancer, or metastatic cancer.

17. The method of claim 13, wherein the disease or condition of bone growth is selected from at least one of: metabolic disorders selected from type 2 diabetes, impaired glucose tolerance, syndrome X, insulin resistance induced by trauma, burns or nitrogen imbalance; adipose tissue disorders (obesity); muscle and neuromuscular disorders such as muscular dystrophy (Duchenne muscular dystrophy); amyotrophic lateral sclerosis (ALS); muscle atrophy; organ atrophy; frailty; carpal tunnel syndrome; congestive obstructive pulmonary disease; and sarcopenia, cachexia and other muscle wasting syndromes; osteoporosis, osteoporosis in elderly and/or postmenopausal women; glucocorticoid-induced osteoporosis; osteopenia; osteoarthritis; and osteoporosis-related fractures; low bone mass due to chronic glucocorticoid therapy, premature gonadal failure, androgen suppression, vitamin D deficiency, secondary hyperparathyroidism, nutritional deficiencies, and anorexia nervosa.

18. The method of claim 13, wherein the muscle-related diseases or conditions are selected from at least one of: neuromuscular disorders, muscular dystrophy and muscle atrophy, congestive obstructive pulmonary disease, muscle wasting associated with COPD, muscle wasting syndrome, sarcopenia, cachexia, adipose tissue disorders, type 2 diabetes, bone degenerative disease, osteoporosis; musculodegenerative and neuromuscular disorders, tissue repair, wound healing, neurodegenerative diseases, amyotrophic lateral sclerosis, immunologic disorders, disorders related to abnormal proliferation or function of lymphocytes, and obesity or disorders related to abnormal proliferation of adipocytes.

19. The method of claim 13, wherein the bispecific antibody or antigen binding fragment having a predetermined activity is used during a first phase of treatment, and a second bispecific antibody or antigen binding fragment having a predetermined activity is used during a second phase of treatment, and optionally using one or more different bispecific antibody or antigen binding fragment having a predetermined activity is used during a first phase of treatment for any additional phases of treatment.

20. A method of selecting a bispecific anti-myostatin and anti-Activin A antibody or antigen binding fragments comprising:
generating the bispecific anti-myostatin and anti-Activin A antibody or antigen binding fragments;
contacting the bispecific anti-myostatin and anti-Activin A antibody or antigen binding fragments under conditions in which a muscle cell expresses myostatin and Activin A;
measuring the effect of the bispecific anti-myostatin and anti-Activin A antibody or antigen binding fragments on the muscle cell; and
selecting the combination of bispecific anti-myostatin and anti-Activin A antibody or antigen binding fragments having a predetermined activity level.

21. The method of claim 20, wherein the activity is selected from at least one of: cachexia, loss of body weight, loss of muscle mass, or loss of fat mass;
gonadal tumor, ovarian cancer, benign prostatic hyperplasia, prostate intraepithelial neoplasia, or prostate cancer, or wherein the tumor is bladder cancer, Wilm's tumor, pancreatic cancer, breast cancer, bone cancer, lung cancer, colorectal cancer, cervical cancer, synovial sarcoma, vasoactive intestinal peptide secreting tumors, glioblastoma, medulloblastoma, head and neck squamous cell cancer, oral cancer, oral leukoplakia, anal cancer, esophageal cancer, gastric cancer, bone cancer, or metastatic cancer;
wherein the disease or condition of bone growth is selected from at least one of: metabolic disorders selected from type 2 diabetes, impaired glucose tolerance, syndrome X, insulin resistance induced by trauma, burns or nitrogen imbalance; adipose tissue disorders (obesity); muscle and neuromuscular disorders such as muscular dystrophy (Duchenne muscular dystrophy); amyotrophic lateral sclerosis (ALS); muscle atrophy; organ atrophy; frailty; carpal tunnel syndrome; congestive obstructive pulmonary disease; and sarcopenia, cachexia and other muscle wasting syndromes; osteoporosis, osteoporosis in elderly and/or postmenopausal women; glucocorticoid-induced osteoporosis; osteopenia; osteoarthritis; and osteoporosis-related fractures; low bone mass due to chronic glucocorticoid therapy, premature gonadal failure, androgen suppression, vitamin D deficiency, secondary hyperparathyroidism, nutritional deficiencies, and anorexia nervosa; or
wherein the muscle-related diseases or conditions are selected from at least one of:
neuromuscular disorders, muscular dystrophy and muscle atrophy, congestive obstructive pulmonary disease, muscle wasting associated with COPD, muscle wasting syndrome, sarcopenia, cachexia, adipose tissue disorders, type 2 diabetes, bone degenerative disease, osteoporosis; musculodegenerative and neuromuscular disorders, tissue repair, wound healing, neurodegenerative diseases, amyotrophic lateral sclerosis, immunologic disorders, disorders related to abnormal proliferation or function of lymphocytes, and obesity or disorders related to abnormal proliferation of adipocytes.

22. The method of claim 20, wherein the bispecific anti-myostatin and anti-Activin A antibody or antigen binding fragments is selected from at least one of:
wherein the bispecific antibody comprises bispecific knob-in-hole, IgG-ScFv, ScFv-IgG, scFV-Fc-ScFv, or scFv-scFv-Fc; or
wherein the bispecific antibody comprises:
a heavy chain variable domain (VH) complementarity determining region 1 (CDR1), CDR2, and CDR3 comprising the amino acid sequence of any one of the following SEQ ID NOs: 3, 4, 5; and 13, 14, 15; and
a light chain variable domain (VL) CDR1, CDR2, and CDR3 comprising the amino acid sequence of any one of the following SEQ ID NOs: 6, 7 and 8; and 16, 17, and 18; or
wherein the bispecific anti-myostatin and anti-Activin A antibody or antigen binding fragments thereof comprises:
a heavy chain comprises at least 80%, or at least 85%, or at least 90%, 91%, 92%, 93%, 94%, 95%, 96%, 97%, 98%, 99% or 100% to SEQ ID NOs: 1 and 2, and
a light chain comprises at least 80%, or at least 85%, or at least 90%, 91%, 92%, 93%, 94%, 95%, 96%, 97%, 98%, 99% or 100% to SEQ ID NOs: 11 and 12; or
wherein the bispecific anti-myostatin and anti-Activin A antibody or antigen binding fragments antibody or binding fragment thereof is encoded by a nucleic acid that comprises:
a VH comprising a nucleic acid sequence that comprises at least 80%, or at least 85%, or at least 90%, 91%, 92%, 93%, 94%, 95%, 96%, 97%, 98%, 99% or 100% to SEQ ID NOS: 9 and 19, and
a VL comprising a nucleic acid sequence that comprises at least 80%, or at least 85%, or at least 90%, 91%, 92%, 93%, 94%, 95%, 96%, 97%, 98%, 99% or 100% to SEQ ID NOs: 10 and 20; or
wherein the bispecific anti-myostatin and anti-Activin A antibody or antigen binding fragments comprises:
a heavy chain variable domain (VH) comprising SEQ ID NOS: 21, 23, or 29; and
a light chain variable domain (VL) comprising SEQ ID NO: 22, 24, and 30.


SEQUENCE LISTING (34 sequences)

SEQ ID NO: 1 - protein, 113 aa, synthetic construct
EVQLVESGGGLVQPGGSLRLSCAASGLTFSRYPMSWVRQAPGKGLVWVSAITSSGGSTYYSDTVKGRFTISRDNAKNTLYLQMNSLRAEDTAVYYCARLPDYWGQGTLVTVSS

SEQ ID NO: 2 - protein, 108 aa, synthetic construct
EIVLTQSPGTLSLSPGERATLSCRASSSVSSSYLHWYQQKPGQAPRLLIYATSNLVAGIPDRFSGSGSGTDFTLTISRLEPEDFAVYYCQHFSGYHFTFGGGTKVEIK

SEQ ID NO: 3 - protein, 8 aa, synthetic construct
GLTFSRYP

SEQ ID NO: 4 - protein, 8 aa, synthetic construct
ITSSGGST

SEQ ID NO: 5 - protein, 6 aa, synthetic construct
ARLPDY

SEQ ID NO: 6 - protein, 7 aa, synthetic construct
SSVSSSY

SEQ ID NO: 7 - skipped

SEQ ID NO: 8 - protein, 9 aa, synthetic construct
QHFSGYHFT

SEQ ID NO: 9 - DNA, 339 bp, synthetic construct
  source 1..339: mol_type = other DNA
gaggtgcagctggtcgaatccggcgggggattagtccaaccaggaggctccctgagactctcttgcgccgcatcagggttgaccttctcacgctatcctatgtcttgggtgaggcaggctcccgggaaaggtttggtgtgggtgtcagctattacaagtagcgggggcagcacatactattctgacacggtgaagggccgatttacaatcagcagggacaatgccaagaacaccctgtacctccagatgaacagtcttcgggcagaggatactgccgtttactattgtgcccgtctaccggattactggggacagggcactctggttaccgtatcgtcc

SEQ ID NO: 10 - DNA, 324 bp, synthetic construct
  source 1..324: mol_type = other DNA
gaaatcgttcttacccagtctccagggacgttatctctgagcccaggagagagggcaacactaagctgtcgagcgagttccagtgtaagctctagctacctgcattggtaccagcagaagcccggccaagctcctcggttgctcatttacgctactagtaacctggtggccggtatccctgaccgcttctcagggtcaggctcaggaacagatttcacactcaccatatccagactggagcccgaagactttgccgtctattattgccagcatttctccgggtatcactttactttcggaggcggcaccaaggtggagattaaa

SEQ ID NO: 11 - protein, 124 aa, synthetic construct
QVQLVESGGGVVQPGRSLRLSCAASGFTFSSYGMHWVRQAPGKGLEWVAVIWYDGSNKYHADSVKGRFTISRDNSKNTLYLQVNSLRAEDTAVYYCVRSRNWNYDNYYYGLDVWGQGTTVTVSS

SEQ ID NO: 12 - protein, 107 aa, synthetic construct
DIQMTQSPSSLSASVGDRVTITCRASQGIRNNLGWYQQKPGKAPKRLIYAASSLQSGVPSRFSGSGSGTEFTLTISSLQPEDFTTYYCLQHNSYPWTFGQGTKVEIK

SEQ ID NO: 13 - protein, 8 aa, synthetic construct
GFTFSSYG

SEQ ID NO: 14 - protein, 8 aa, synthetic construct
IWYDGSNK

SEQ ID NO: 15 - protein, 17 aa, synthetic construct
VRSRNWNYDNYYYGLDV

SEQ ID NO: 16 - protein, 7 aa, synthetic construct
QGIRNNL

SEQ ID NO: 17 - skipped

SEQ ID NO: 18 - protein, 9 aa, synthetic construct
LQHNSYPWT

SEQ ID NO: 19 - DNA, 372 bp, synthetic construct
  source 1..372: mol_type = other DNA
caggtccagctcgtcgaatccggtggaggcgtcgtgcagccaggacggtcactcaggttgtcctgtgcagcttcaggattcactttttcctcatatggaatgcactgggtaagacaagctccggggaaaggcttggagtgggtggctgtaatctggtacgatggaagcaataaatatcatgcagacagtgtgaaagggagattcactatctccagggataactccaaaaataccctgtatcttcaagtgaacagcctgcgcgctgaggacaccgcggtgtactactgtgtccggtcaaggaactggaattacgataactattattacgggttggacgtttggggacaaggcaccactgtgactgtgtcaagt

SEQ ID NO: 20 - DNA, 321 bp, synthetic construct
  source 1..321: mol_type = other DNA
gacattcaaatgacccagagtcccagctccctgtccgccagtgtcggtgacagagtgacgataacttgccgcgctagccagggcatacgcaataatctgggatggtaccagcaaaaacccgggaaggcacctaagcggctcatctacgctgcaagctcacttcagtccggggtcccaagtcgattcagtgggagtggaagtggtacggaattcactcttacaatatcttccctgcagccggaagacttcactacgtactactgtcttcagcacaattcttacccctggacatttggccagggcacaaaggtcgagatcaaa

SEQ ID NO: 21 - protein, 454 aa, synthetic construct
QVQLVESGGGVVQPGRSLRLSCAASGFTFSSYGMHWVRQAPGKGLEWVAVIWYDGSNKYHADSVKGRFTISRDNSKNTLYLQVNSLRAEDTAVYYCVRSRNWNYDNYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 22 - protein, 214 aa, synthetic construct
DIQMTQSPSSLSASVGDRVTITCRASQGIRNNLGWYQQKPGKAPKRLIYAASSLQSGVPSRFSGSGSGTEFTLTISSLQPEDFTTYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 443 aa, synthetic construct
EVQLVESGGGLVQPGGSLRLSCAASGLTFSRYPMSWVRQAPGKGLVWVSAITSSGGSTYYSDTVKGRFTISRDNAKNTLYLQMNSLRAEDTAVYYCARLPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 24 - protein, 215 aa, synthetic construct
EIVLTQSPGTLSLSPGERATLSCRASSSVSSSYLHWYQQKPGQAPRLLIYATSNLVAGIPDRFSGSGSGTDFTLTISRLEPEDFAVYYCQHFSGYHFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - DNA, 1362 bp, synthetic construct
  source 1..1362: mol_type = other DNA
caggtccagctcgtcgaatccggtggaggcgtcgtgcagccaggacggtcactcaggttgtcctgtgcagcttcaggattcactttttcctcatatggaatgcactgggtaagacaagctccggggaaaggcttggagtgggtggctgtaatctggtacgatggaagcaataaatatcatgcagacagtgtgaaagggagattcactatctccagggataactccaaaaataccctgtatcttcaagtgaacagcctgcgcgctgaggacaccgcggtgtactactgtgtccggtcaaggaactggaattacgataactattattacgggttggacgtttggggacaaggcaccactgtgactgtgtcaagtgcatccaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagagagtggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacgcgagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcctgctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 26 - DNA, 642 bp, synthetic construct
  source 1..642: mol_type = other DNA
gacattcaaatgacccagagtcccagctccctgtccgccagtgtcggtgacagagtgacgataacttgccgcgctagccagggcatacgcaataatctgggatggtaccagcaaaaacccgggaaggcacctaagcggctcatctacgctgcaagctcacttcagtccggggtcccaagtcgattcagtgggagtggaagtggtacggaattcactcttacaatatcttccctgcagccggaagacttcactacgtactactgtcttcagcacaattcttacccctggacatttggccagggcacaaaggtcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 27 - DNA, 1329 bp, synthetic construct
  source 1..1329: mol_type = other DNA
gaggtgcagctggtcgaatccggcgggggattagtccaaccaggaggctccctgagactctcttgcgccgcatcagggttgaccttctcacgctatcctatgtcttgggtgaggcaggctcccgggaaaggtttggtgtgggtgtcagctattacaagtagcgggggcagcacatactattctgacacggtgaagggccgatttacaatcagcagggacaatgccaagaacaccctgtacctccagatgaacagtcttcgggcagaggatactgccgtttactattgtgcccgtctaccggattactggggacagggcactctggttaccgtatcgtccgcgtcaacaaaaggtccctcagtgtttcctctggccccatcaagcaagagcacctccggcggcaccgccgcactgggatgtctcgtgaaggattacttccctgagcctgtaactgtgagctggaattctggtgcgctgacaagtggtgtccataccttccccgccgtcttgcagagcagtgggctgtatagcttaagcagcgtagtgaccgtccctagctcctccctgggtactcagacttatatctgcaatgtcaatcataaaccctctaataccaaagtcgataaacgcgtggagcctaaatcttgtgataaaacccatacctgcccaccatgtcccgcccctgagctgctcggcggtccatccgtcttcctgtttcctccaaaaccaaaggacacattgatgattagtaggacacccgaggtcacctgtgtggtggttgacgtttcccacgaggacccagaggtcaaattcaactggtatgtggacggcgtggaggtccacaacgctaaaaccaaacccagagaagaacagtacgcctctacttatagagttgtgagcgtcctcacggtgctgcaccaggattggctgaatggcaaggagtataagtgcaaagtctctaataaagcactccctgcgccaatagagaagacaattagcaaggcaaagggacaaccacgcgaacctcaggtgtacactctcccccctagccgggaagagatgacaaaaaatcaggtttctttaacatgtctggtgaaaggattttacccttccgacatcgcggtcgaatgggagtcaaatggacaacccgagaataattacaagacaacaccaccagtgttagactccgatggctccttctttctgtacagcaggctgacggtggacaagagccgctggcaacaggggaatgtcttctcttgctctgtgatgcatgaggcactccacaatcattatacgcagaaaagtctctctctctctccaggcaaa

SEQ ID NO: 28 - DNA, 324 bp, synthetic construct; identical to SEQ ID NO: 10
  source 1..324: mol_type = other DNA

SEQ ID NO: 29 - protein, 701 aa, synthetic construct
QVQLVESGGGVVQPGRSLRLSCAASGFTFSSYGMHWVRQAPGKGLEWVAVIWYDGSNKYHADSVKGRFTISRDNSKNTLYLQVNSLRAEDTAVYYCVRSRNWNYDNYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGGGGGSGGGGSGGGGSEVQLVESGGGLVQPGGSLRLSCAASGLTFSRYPMSWVRQAPGKGLVWVSAITSSGGSTYYSDTVKGRFTISRDNAKNTLYLQMNSLRAEDTAVYYCARLPDYWGQGTLVTVSSGGGGSGGGGSGGGGSEIVLTQSPGTLSLSPGERATLSCRASSSVSSSYLHWYQQKPGQAPRLLIYATSNLVAGIPDRFSGSGSGTDFTLTISRLEPEDFAVYYCQHFSGYHFTFGGGTKVEIK

SEQ ID NO: 30 - protein, 214 aa, synthetic construct; identical to SEQ ID NO: 22

SEQ ID NO: 31 - DNA, 2106 bp, synthetic construct
  source 1..2106: mol_type = other DNA
caggtacaattggtagagagcggaggaggcgttgtgcagccaggtcggtctctccgactgtcctgtgccgcctctggcttcacgttctcctcatatggaatgcattgggtccgtcaagctccagggaaaggactcgagtgggtcgccgtcatttggtacgatggtagcaataagtatcatgccgatagcgtcaagggcagatttaccatcagtagagacaacagtaagaacactttgtatctgcaagtgaatagtctgcgggcagaggacacggcagtgtactactgtgtaaggtctaggaattggaattatgacaactactattatggcctcgatgtttggggccaggggacaactgtcaccgtctcatccgctagtacaaaaggacctagcgtgttccctttggctccctgtagtcgttcaacctctgaaagcacagcagcgctggggtgtttggtcaaggactattttcccgaacccgtgaccgtcagctggaattctggggcgttgacctctggagttcatacatttcccgctgtgctccaaagttccggcttgtattcccttagtagcgttgttaccgttccatcatccagcttgggcaccaaaacttatacctgtaatgttgatcataaaccctcaaatacaaaggtcgataagagggtcgaaagcaaatacgggcccccttgtccgccctgtccagcgcccgagtttctgggcgggccttctgtgttcttgttcccgccaaaacccaaagatactctcatgattagccgaactcccgaggtgacatgcgtcgtcgttgatgtctctcaagaagatcctgaagtgcaatttaattggtatgttgatggcgtggaagtacacaatgctaagaccaagccccgcgaggaacaatttaatagcacttaccgtgtggtgagcgttctcactgtcctgcatcaagactggttgaatggtaaagaatataaatgcaaagtgtccaataaaggactgcccagcagcatcgaaaagaccatatccaaagctaaggggcaaccgcgagaacctcaagtttatacgctcccaccatcccaagaagaaatgacaaagaaccaagtgtcccttacttgcttggtgaagggcttttacccttcagatatagcagtagaatgggaaagcaatggccaacctgaaaacaattataaaacaactcccccagtactggatagtgatggctctttcttcctttatagccgtcttacagttgacaagagccgctggcaagaaggcaatgtgttctcttgttcagtcctgcacgaagcgctccactctcattatactcagaagagtttgtcactcagtttggggggtggtggcggttctggtggtggtggctctggtggcggcggttctgaagtccagctggttgaatccggcggtggtctcgtccagccgggtggatctctgaggttgagctgtgctgcaagcggtctgaccttttctcgatacccgatgtcctgggtccgccaagctccagggaaaggtctggtgtgggtttcagccattacgagctccggcggctcaacctactatagtgataccgtgaagggtagattcacgatctccagagacaatgctaagaataccctgtatcttcaaatgaattctctgagggctgaggatacagctgtttattattgtgcaaggctccctgactattggggacaaggcacactcgtcacagtctcaagtggcggtggtggttctggcggcggtggaagcggcggtggcggaagcgaaatcgtgttgacccagtccccaggcactctgtctctgtctccaggtgagcgagcaaccctgtcttgcagggcttcatcatccgtgagttcttcctacctgcactggtaccaacaaaagcctggacaggcccctcgtctcctcatctacgctacatccaatctcgttgctggtattccagaccgattttctggctccgggagcggcacagattttactttgacaatctctcggctggaacccgaggacttcgccgtgtattattgtcaacacttcagcggatatcattttacctttggtggtggaacaaaagtggaaatcaaataa

SEQ ID NO: 32 - DNA, 642 bp, synthetic construct; identical to SEQ ID NO: 26
  source 1..642: mol_type = other DNA

SEQ ID NO: 33 - protein, 5 aa, synthetic construct
GGGGS

SEQ ID NO: 34 - protein, 45 aa, synthetic construct
GGGGGPROTEINSYNTHETICCONSTRUCTGGGGSGGGGSGGGGS